(19) United States
(12) Patent Application Publication
(10) Pub. No.: US 2024/0025954 A1
(43) Pub. Date: Jan. 25, 2024

(54) Compositions and Methods for the Treatment of Alzheimer's Disease

(71) Applicant: SOLA BIOSCIENCES LLC, Natick, MA (US)

(72) Inventors: Akinori Hishiya, Belmont, MA (US); Keizo Koya, Chestnut Hill, MA (US)

(21) Appl. No.: 18/033,454

(22) PCT Filed: Oct. 26, 2021
(86) PCT No.: PCT/US21/56539

§ 371 Date: Apr. 24, 2023

Related U.S. Application Data
(60) Provisional application No. 63/105,472, filed on Oct. 26, 2020.

(51) Int. Cl. C07K 14/47; C12N 15/86; C07K 16/18; A61P 25/28

(52) U.S. Cl. CPC C07K 14/47; C12N 15/86; C07K 16/18; A61P 25/28; C12N 2750/14143; C07K 2319/30

(57) ABSTRACT

A novel class of fusion proteins to recruit a cell's innate chaperone mechanism, specifically the Hsp70-mediated system, to specifically reduce tau-mediated protein aggregation and associated proteopathies is disclosed.

### CROSS REFERENCE TO RELATED APPLICATION

This application claims priority under 35 U.S.C. § 119(e) to the U.S. Provisional Patent Application 63/105,472 filed Oct. 26, 2020. The entire contents of the aforementioned application is hereby incorporated by reference in its entirety.

### BACKGROUND

All proteins expressed within a cell need to correctly fold into their intended structures in order to function properly. A growing number of diseases and disorders are shown to be associated with inappropriate folding of proteins and/or inappropriate deposition and aggregation of proteins and lipoproteins as well as infectious proteinaceous substances. Also known as a conformational disease or proteopathy, examples of diseases caused by misfolding include Alzheimer's disease (AD), amyotrophic lateral sclerosis (ALS), and frontotemporal lobar dementia (FTLD). The mutant protein aggregates in cells causing typical cytotoxic cellular inclusion bodies.

A wide variety of neurodegenerative diseases are characterized pathologically by the accumulation of intracellular or extracellular protein aggregates composed of amyloid fibrils (Forman et al., (2004) Nat Med 10:1055-1063).

AD is the most common neurodegenerative disorder affecting one in eight older Americans. Approximately 32% of people over 85 years old suffer the disease with more than 5.4 million patients in the US alone. Despite intensive research, there is no cure for AD, and only symptomatic treatment is available.

AD is characterized by memory deficits and overall cognitive dysfunctions due to synaptic loss and neuronal death in certain brain regions including the hippocampus and neocortical brain (Norfray & Provenzale, (2004) AJR Am J Roentgenol 182:3-13; Ittner & Gotz (2011) Nat Rev Neurosci 12:65-72). Although it's now clear that neuronal degeneration correlates with AD memory and cognitive impairments, the molecular mechanisms underlying neurodegeneration in affected regions remain largely unexplained. The neuronal damages are associated with deposition of amyloid β (Aβ) in extracellular senile plaques (Selkoe, (2001) Neuron 32:177-180; Fan et al., (2007) J Neuroinflammation 4:22), and intraneuronal neurofibrillary tangles (NFTs) consisting of hyperphosphorylated tau proteins. (Ittner & Gotz (2011) Nat Rev Neurosci 12:65-72; Francis et al., (1999) J Neural Neurosurg Psychiatry 66:137-147; Craig et al., (2011) Neurosci Biobehav Rev 35:1397-1409). The contribution of these protein deposits to AD pathology is, however, largely unknown.

Both Aβ and tau may have normal roles at the synapse while pathological species of these proteins can contribute to synapse degeneration (Spires-Jones & Hyman, (2014) Neuron 82:756-771).

In addition to the histological studies, the discoveries of genetic linkages for early-onset familial AD (FAD) to several loci provide promises to identify genetic factors that contribute to the pathogenesis of the disease. Early-onset FAD is caused by hereditary genetic mutations in three genes (APP, PSEN1, and PSEN2) and one significant genetic risk factor, the APOEε4 allele (Spires-Jones & Hyman, (2014) Neuron 82:756-771). Additional genetic risk loci have been identified in genome-wide association studies (GWAS) and massive parallel resequencing (MPS) efforts (Van Cauwenberghe et al., (2016) Genet Med 18:421-430).

These genetic studies have not revealed the correlation between tau and FAD, however, linkage analyses have suggested a correlation between tau and other neurodegenerative diseases, many of which were mapped to the region 17q21-22 on chromosome where the tau gene is located (Wilhelmsen et al., (1994) Am J Hum Genet 55:1159-1165; Wijker et al., (1996) Hum Mol Genet 5:151-154; Bird et al., (1997) Neurology 48:949-954; Lendon et al., (1998) Neurology 50:1546-1555). Subsequently, mutations in the tau gene were identified from hereditary dominant frontotemporal dementia with parkinsonism in chromosome 17 (FTDP-17) (Foster et al., (1997) Ann Neural 41:706-715; Hutton et al., (1998) Nature 393:702-705; Poorkaj et al., (1998) Ann Neural 43:815-825; Spillantini et al., (1998) Proc Natl Acad Sci USA 95:7737-7741).

Tau is a microtubule-associated protein that binds to microtubules and promotes microtubule assembly. The binding of tau to microtubules promotes microtubule polymerization (Cleveland et al., (1977)J Mol Biol 116:227-247; Weingarten et al., (1975) Proc Natl Acad Sci USA 72:1858-1862). Analysis of tau sequence revealed that tau has four microtubule binding motifs that are located in 4 repeat regions at the C-terminal end of the protein with conserved 18-amino acid long binding elements separated by less conserved 13-14 amino acids (Lee et al., (2001) Neurosci 24:1121-1159). In the adult human brain, six tau isoforms ranging from 325 to 441 amino acids in length are produced by alternative splicing (Goedert et al., (1988) Proc Natl Acad Sci USA 85:4051-4055).

Tau is abundantly expressed in the central nervous system (CNS), and predominantly located in axons, while tau is expressed at lower levels in axons of the peripheral nervous system (PNS). Only low levels of expression are observed in CNS astrocytes and oligodendrocytes (Cleveland et al., (1977) J Mol Biol 116:227-247; Binder et al., (1985) J Cell Biol 101:1371-1378; LoPresti et al., (1995) Proc Natl Acad Sci US A 92:10369-10373).

Tau is an intrinsically disordered and highly soluble protein under normal condition (Jeganathan et al., (2008) Biochemistry 47:10526-10539), while it turns insoluble through aberrant modifications such as hyperphosphorylation (Kopeikina et al., (2012) Transl Neurosci 3:223-233). Hyperphosphorylated tau proteins can result in the self-assembly of tangles of paired helical filament (PHF) and straight filaments, which are involved in the pathogenesis of AD, frontotemporal dementia (FTD) and other tauopathies. Paired helical filament (PHF) and hyperphosphorylation of tau proteins are two of the most recognized molecular characteristics in AD and in several other neurodegenerative tauopathies including FTDP-17 and progressive supranuclear palsy (PSP) (Lee et al., (2001) Neurosci 24:1121-1159).

Tauopathies are a group of neurodegenerative disorders that share a common pathological feature, formation of insoluble intraneuronal aggregates composed of filamentous hyperphosphorylated tau proteins (Lee et al., (2001) Neurosci 24:1121-1159; Delacourte, (2001) Adv Exp Med Biol 487:5-19).

Despite their diverse clinical manifestations, these neurodegenerative disorders share a common pathological feature: formation of insoluble intraneuronal aggregates composed of filamentous hyperphosphorylated tau proteins (Lee et al., (2001) Neurosci 24:1121-1159; Delacourte, (2001) Adv Exp Med Biol 487:5-19), implicating tau dysfunction as a contributing factor in these neurodegenerative diseases (Lee et al., (2001) Neurosci 24:1121-1159). Some mutations in the tau gene lead to the formation of filaments made of hyperphosphorylated tau protein (Crowther & Goedert, (2000) J Struct Biol 130:271-279).

The heat shock 70 kDa proteins (referred to herein as “Hsp70s”) constitute a ubiquitous class of chaperone proteins in the cells of a wide variety of species (Tavaria et al., (1996) Cell Stress Chaperones 1:23-28). Hsp70 requires assistant proteins called co-chaperone proteins, such as J domain proteins and nucleotide exchange factors (NEFs) (Hartl et al., (2009) Nat Struct Mol Biol 16:574-581), in order to function. In the current model of Hsp70 chaperone machinery for folding proteins, Hsp70 cycles between ATP- and ADP-bound states, and a J domain protein binds to another protein (referred to as a “client protein”) in need of folding or refolding, interacting with the ATP-bound form of Hsp70 (Hsp70-ATP) (Young (2010) Biochem Cell Biol 88:291-300; Mayer, (2010) Mol Cell 39:321-331). Binding of the J domain protein-client complex to Hsp70-ATP stimulates ATP hydrolysis, which causes a conformational change in the Hsp70 protein, closing a helical lid and, thereby, stabilizing the interaction between the client protein with Hsp70-ADP, as well as eliciting the release of the J domain protein that is then free to bind to another client protein.

Therefore, according to this model, J domain proteins play a critical role within the Hsp70 machinery by acting as a bridge, and facilitating the capture and submission of a wide variety of client proteins into the Hsp70 machinery to promote proper folding or refolding (Kampinga & Craig (2010) Nat Rev Mol Cell Biol 11:579-592). The J domain family is widely conserved in species ranging from prokaryotes (DnaJ protein) to eukaryotes (Hsp40 protein family). The J domain (about 60-80 aa) is composed of four helices: I, II, III, and IV. Helices II and III are connected via a flexible loop containing an “HPD motif”, which is highly conserved across J domains and thought to be critical for activity since mutations within the HPD sequence abolish J domain function (Tsai & Douglas, (1996) J Biol Chem 271:9347-9354).

Given the context provided above for proteopathies such as AD, it seems clear that reducing the level of misfolded proteins could serve as a means to treat, prevent or otherwise ameliorate the symptoms of these devastating disorders and that, recruitment of a cell's innate ability to repair protein misfolding would be a logical choice to pursue.

### SUMMARY OF THE INVENTION

The inventors have developed a novel class of fusion proteins to recruit a cell's innate Hsp70-mediated chaperone mechanism, to specifically reduce tau-mediated protein aggregation. Unlike in previous studies by the inventors using fusion proteins comprising fragments of a Hsp40 protein (also called J proteins), a co-chaperone that interacts with Hsp70, to enhance protein secretion and expression, the present study employs J domain-containing fusion proteins for the purpose of reducing protein aggregation and cytotoxicity caused by aggregation of mutant tau proteins. In this context, the inventors have made the surprising discovery that the elements of the J domain required for function is quite distinct from use of J domains in enhancing protein expression and secretion, demonstrating a distinct mechanism for the mode of action of the present fusion proteins. The fusion proteins described herein comprise a J domain and a domain that has affinity for tau. The presence of the tau-binding domain within the fusion protein results in specific reduction in aggregation of mutant tau proteins.
E1. Therefore, in a first aspect, disclosed herein is an isolated fusion protein comprising a J domain of a J protein and a tau-binding domain.
E2. The fusion protein of E1, wherein the J domain of a J protein is of eukaryotic origin.
E3. The fusion protein of any of E1-E2, wherein the J domain of a J protein is of human origin.
E4. The fusion protein of any of E1-E3, wherein the J domain of a J protein is cytosolically localized.
E5. The fusion protein of any of E1-E4, wherein the J domain of a J protein is selected from the group consisting of SEQ ID Nos: 1-48.
E6. The fusion protein of any of E1-E5, wherein the J domain comprises the sequence selected from the group consisting of SEQ ID NOs: 5, 6, 10, 24, and 31.
E7. The fusion protein of any of E1-E6, wherein the J domain comprises the sequence of SEQ ID NO: 5.
E8. The fusion protein of any of E1-E6, wherein the J domain comprises the sequence of SEQ ID NO: 6.
E9. The fusion protein of any of E1-E6, wherein the J domain comprises the sequence of SEQ ID NO: 10.
E10. The fusion protein of any of E1-E6, wherein the J domain comprises the sequence of SEQ ID NO: 24.
E11. The fusion protein of any of E1-E6, wherein the J domain comprises the sequence of SEQ ID NO:31.
E12. The fusion protein of any of E1-E11, wherein the tau-binding domain has a K_{D} for tau of 1 μM or less, for example, 300 nM or less, 100 nM or less, 30 nM or less, 10 nM or less when measured using an ELISA assay.
E13. The fusion protein of any of E1-E12, wherein the tau-binding domain comprises the sequence selected from the group consisting of SEQ ID NOs: 49-54.
E14. The fusion protein of any of E1-E13, wherein the tau-binding domain comprises the sequence of SEQ ID NO:49.
E15. The fusion protein of any of E1-E13, wherein the tau-binding domain comprises the sequence of SEQ ID NO:50.
E16. The fusion protein of any of E1-E13, wherein the tau-binding domain comprises the sequence of SEQ ID NO:51.
E17. The fusion protein of any of E1-E13, wherein the tau-binding domain comprises the sequence of SEQ ID NO:52.
E18. The fusion protein of any of E1-E13, wherein the tau-binding domain comprises the sequence of SEQ ID NO:53.
E19. The fusion protein of any of E1-E13, wherein the tau-binding domain comprises the sequence of SEQ ID NO:54.
E20. The fusion protein of any of E1-E19, comprising a plurality of tau-binding domains.
E21. The fusion protein of any of E1-E20, consisting of two tau-binding domains.
E22. The fusion protein of any of E1-E21, consisting of three tau-binding domains.
E23. The fusion protein of any of E1-E22, comprising one of the following constructs:

| | |
|---|---|
| a. | DNAJ-X-T, |
| b. | DNAJ-X-T-X-T, |
| c. | DNAJ-X-T-X-T-X-T, |
| d. | T-X-DNAJ, |
| e. | T-X-T-X-DNAJ, |
| f. | T-X-T-X-T-X-DNAJ, |
| g. | T-X-DNAJ-X-T, |
| h. | T-X-DNAJ-X-T-X-T, |
| i. | T-X-DNAJ-X-T-X-T-X-T, |
| j. | T-X-T-X-DNAJ-X-T, |
| k. | T-X-T-X-DNAJ-X-T-X-T, |
| l. | T-X-T-X-DNAJ-X-T-X-T-X-T, |
| m. | T-X-T-X-T-X-DNAJ-X-T, |
| n. | T-X-T-X-T-X-DNAJ-X-T-X-T, and |
| o. | T-X-T-X-T-X-DNAJ-X-T-X-T-X-T, |

wherein,
T is a tau-binding domain,
DNAJ is a J domain of a J protein, and
X is an optional linker.
E24. The fusion protein of any of E1-E23, wherein the fusion protein comprises the J domain sequence of SEQ ID NO: 5 and the tau-binding domain sequence of SEQ ID NO: 49.
E25. The fusion protein of any of E1-E23, wherein the fusion protein comprises the J domain sequence of SEQ ID NO: 5 and the tau-binding domain sequence of SEQ ID NO: 50.
E26. The fusion protein of any of E1-E23, wherein the fusion protein comprises the J domain sequence of SEQ ID NO: 5 and the tau-binding domain sequence of SEQ ID NO: 51.
E27. The fusion protein of any of E1-E23, wherein the fusion protein comprises the J domain sequence of SEQ ID NO: 5 and the tau-binding domain sequence of SEQ ID NO: 52.
E28. The fusion protein of any of E1-E23, wherein the fusion protein comprises the J domain sequence of SEQ ID NO: 5 and the tau-binding domain sequence of SEQ ID NO:53.
E29. The fusion protein of any of E1-E23, wherein the fusion protein comprises the J domain sequence of SEQ ID NO: 5 and the tau-binding domain sequence of SEQ ID NO:54.
E30. The fusion protein of any of E1-E29, wherein the fusion protein comprises the sequence selected from the group consisting of SEQ ID NOs: 83-88 and 95-101.
E31. The fusion protein of any of E1-E30, wherein the fusion protein comprises the sequence of SEQ ID NO: 83.
E32. The fusion protein of any of E1-E30, wherein the fusion protein comprises the sequence of SEQ ID NO: 84.
E33. The fusion protein of any of E1-E30, wherein the fusion protein comprises the sequence of SEQ ID NO: 85.
E34. The fusion protein of any of E1-E30, wherein the fusion protein comprises the sequence of SEQ ID NO: 86.
E35. The fusion protein of any of E1-E30, wherein the fusion protein comprises the sequence of SEQ ID NO: 87.
E36. The fusion protein of any of E1-E30, wherein the fusion protein comprises the sequence of SEQ ID NO: 88.
E37. The fusion protein of any of E1-E30, wherein the fusion protein comprises the sequence of SEQ ID NO:95.
E38. The fusion protein of any of E1-E30, wherein the fusion protein comprises the sequence of SEQ ID NO:96.
E39. The fusion protein of any of E1-E30, wherein the fusion protein comprises the sequence of SEQ ID NO:97.
E40. The fusion protein of any of E1-E30, wherein the fusion protein comprises the sequence of SEQ ID NO:98.
E41. The fusion protein of any of E1-E30, wherein the fusion protein comprises the sequence of SEQ ID NO:99.
E42. The fusion protein of any of E1-E30, wherein the fusion protein comprises the sequence of SEQ ID NO:100.
E43. The fusion protein of any of E1-E30, wherein the fusion protein comprises the sequence of SEQ ID NO:101.
E44. The fusion protein of any of E1-E43, further comprising a targeting reagent.
E45. The fusion protein of any of E1-E44, further comprising an epitope.
E46. The fusion protein of E45, wherein the epitope is a polypeptide selected from the group consisting of SEQ ID NOs: 66-72.
E47. The fusion protein of any of E1-E46, further comprising a cell-penetrating agent.
E48. The fusion protein of E47, wherein the cell-penetrating agent comprises a peptide sequence selected from the group consisting of SEQ ID NOs: 73-76.
E49. The fusion protein of any of E1-E48, further comprising a signal sequence.
E50. The fusion protein of E49, wherein the signal sequence comprises the peptide sequence selected from the group consisting of SEQ ID NOs: 77-79.
E51. The fusion protein of any of E1-E50, which is capable of reducing aggregation of tau proteins in a cell.
E52. The fusion protein of any of E1-E51, which is capable of reducing tau-mediated cytotoxicity.
E53. A nucleic acid sequence encoding the fusion protein of any of E1-E52.
E54. The nucleic acid sequence of E53, wherein said nucleic acid is DNA.
E55. The nucleic acid sequence of any of E54, wherein said nucleic acid is RNA.
E56. The nucleic acid sequence of any of E53-E55, wherein said nucleic acid comprises at least one modified nucleic acid.
E57. A vector comprising the nucleic acid sequence of any of E53-E56.
E58. The vector of E57, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.
E59. A virus particle comprising a capsid and the vector of E57 or E58.
E60. The virus particle of E59, wherein the capsid is selected from the group consisting of AAV1, AAV2, AAV3, AAV4, AAV5, AAV6, AAV7, AAV8, AAV9, AAV10, AAVrh10, AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.
E61. A pharmaceutical composition comprising an agent selected from the group consisting of the fusion protein of any of E1-E52, a cell expressing the fusion protein of E1-E52, the nucleic acid of any of E53-E56, the vector of any of E57-E58, the virus particle of any of E59-E60, and a pharmaceutically acceptable carrier or excipient.
E62. A method of reducing toxicity of a tau protein in a cell, comprising contacting said cell with the fusion protein of any of E1-E52, a cell expressing the fusion protein of E1-E52, the nucleic acid of any of E53-E56, the vector of any of E57-E58, the virus particle of any of E59-E60, and the pharmaceutically composition of E61.
E63. The method of E62, wherein the cell is in a subject.
E64. The method of any of E62-E63, wherein the subject is a human.
E65. The method of any one of E62-E64, wherein the cell is a cell of the central nervous system.
E66. The method of any one of E62-E65, wherein subject is identified as having a tauopathy.
E67. The method of E66, wherein the tau disease is selected from the group consisting of Alzheimer's Disease (AD), Parkinson's Disease (PD), Primary age-related tauopathy (PART), Chronic traumatic encephalopathy (CTE), Progressive supranuclear palsy (PSP), Corticobasal degeneration (CBD), Frontotemporal dementia and parkinsonism linked to chromosome 17 (FTDP-17), Lytico-bodig disease, Ganglioglioma and gangliocytoma, Meningioangiomatosis, Postencephalitic parkinsonism, Subacute sclerosing panencephalitis (SSPE), lead encephalopathy, tuberous sclerosis, Pantothenate kinase-associated neurodegeneration, and lipofuscinosis.
E68. The method of E66 or E67, wherein the tauopathy is Alzheimer's Disease (AD).
E69. The method of any one of E62-E68, wherein there is a reduction in the amount of aggregated tau protein in the cell when compared with a control cell.
E70. A method of treating, preventing, or delaying the progression of a tau disease in a subject in need thereof, the method comprising administering an effective amount of one or more agents selected from the group consisting of with the fusion protein of any of E1-E52, a cell expressing the fusion protein of E1-E52, the nucleic acid of any of E53-E56, the vector of any of E57-E58, the virus particle of any of E59-E60, and the pharmaceutically composition of E61.
E71. The method of E70, wherein the tau disease is selected from the group consisting of Alzheimer's Disease (AD), Parkinson's Disease (PD), Primary age-related tauopathy (PART), Chronic traumatic encephalopathy (CTE), Progressive supranuclear palsy (PSP), Corticobasal degeneration (CBD), Frontotemporal dementia and parkinsonism linked to chromosome 17 (FTDP-17), Lytico-bodig disease, Ganglioglioma and gangliocytoma, Meningioangiomatosis, Postencephalitic parkinsonism, Subacute sclerosing panencephalitis (SSPE), lead encephalopathy, tuberous sclerosis, Pantothenate kinase-associated neurodegeneration, and lipofuscinosis.
E72. The method of E70 or E71, wherein the tauopathy is Alzheimer's Disease (AD).
E73. Use of one or more of the fusion protein of any of E1-E52, a cell expressing the fusion protein of E1-E52, the nucleic acid of any of E53-E56, the vector of any of E57-E58, the virus particle of any of E59-E60, and the pharmaceutically composition of E61, in preventing or delaying the progression of a tau disease in a subject.
E74. Use of one or more of the fusion protein of any of E1-E52, a cell expressing the fusion protein of E1-E52, the nucleic acid of any of E53-E56, the vector of any of E57-E58, the virus particle of any of E59-E60, and the pharmaceutically composition of E61, in the preparation of a medicament useful for treating, preventing or delaying the progression of a tauopathy in a subject.

### DESCRIPTION OF THE FIGURES

FIG. 1A shows a Clustal Omega sequence alignment of representative human J domain sequences. The highly conserved HPD domain is shown in the highlighted box.

FIG. 1B shows a Clustal Omega sequence alignment of representative human J domain sequences.

FIG. 2 shows some illustrative fusion protein constructs comprising a J domain and tau-binding domains.

FIG. 3 shows a western blot analysis of cell extracts displaying either wildtype tau ON4R (lanes 2-6) or mutant (P243S) tau (lanes 7-11) expression, each comprising a V5 epitope, and also co-expressing various fusion protein constructs JB1-TBP Construct 1, (lanes 3 and 8); JB1-ScFv(tau) Construct 4 (lanes 4 and 9); JB1-ScFv(MW7) Construct 5, (lanes 5 and 10); and JB1-Happ1 Construct 6, (lanes 6 and 11). The top panel shows levels of tau protein as probed anti-V5 antibodies, while the lower panel shows levels of phosphorylated tau as probed with anti-pTau(Ser396) antibodies.

FIG. 4 shows immunoblot analyses of cell extracts displaying either wildtype tau ON4R (lanes 2, 3, 7 and 8) or mutant (P243S) tau (lanes 4, 5, 9, 10) expression either alone or co-expressing JB1-ScFv(tau) containing a Flag epitope (lanes 3, 5, 8 and 10). The top panel was probed with antibodies recognizing phosphorylated tau at Thr231 (lanes 1-5) or phosphorylated tau at Ser396 in tau 2N4R (lanes 6-10). The bottom panel was probed with anti-Flag antibodies to detect the J domain fusion protein (in this case, the JB1-ScFv(tau) construct, or Construct 4).

FIG. 5 shows immunoblot analyses of cell extracts displaying either wildtype tau ON4R (lanes 2-5) or mutant (P243S) tau (lanes 6-9) expression either alone (lanes 1 and 6) or co-expressing Construct 4 [JB1-ScFv(tau) (lanes 4 and 8), Construct 8 [a control construct which is identical to JB1-ScFv(tau) with the exception of a P33Q mutation within the conserved HPD motif of the J domain (lanes 5 and 9)], and ScFv(tau) only [Construct 4 without the J domain (lanes 3 and 7)].

FIG. 6 shows immunoblots of cell extracts displaying wildtype (lanes 2-6) or mutant (P243S) tau (lanes 7-11) expression either alone (lanes 2 and 7), co-expressing Construct 6 (JB1-Happ1, lanes 3 and 8), Construct 1 (JB1-TBP1, lanes 4 and 9), Construct 14 (JB1-TBP2, lanes 5 and 10) or Construct 11 (JB1-QBP1, lanes 6 and 11).

FIG. 7 shows immunoblots of cell extracts displaying expression of wildtype (lanes 1-3) or truncated Tau3R (lanes 4-6), and wildtype tau or truncated Tau3R co-expressing Construct 1 (JB1-TBP1, lanes 2 and 5) or Construct 7 (JB1-TBP1 containing the P33Q mutation within the conserved J domain HPD motif (lanes 3 and 6).

FIG. 8 shows the effect of expressing Construct 1 (JB1-TBP1) on tau-mediated cytotoxicity, as measured using an LDH assay.

### DEFINITIONS

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other modification, such as conjugation with a labeling component.

As used herein the term “amino acid” refers to either natural and/or unnatural or synthetic amino acids, including but not limited to both the D or L optical isomers, and amino acid analogs and peptidomimetics. Standard single or three letter codes are used to designate amino acids.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “tau disorder”, “tau disease”, “tauopathy” or “tau-mediated disease”, as herein defined refers to disorders associated with formation of intracellular tau aggregates, particularly aggregates of tau mutant protein. Examples of tau disorders include, but are not limited to Alzheimer's Disease (AD), Parkinson's Disease (PD), Primary age-related tauopathy (PART), Chronic traumatic encephalopathy (CTE), Progressive supranuclear palsy (PSP), Corticobasal degeneration (CBD), Frontotemporal dementia and parkinsonism linked to chromosome 17 (FTDP-17), Lytico-bodig disease, Ganglioglioma and gangliocytoma, Meningioangiomatosis, Postencephalitic parkinsonism, Subacute sclerosing panencephalitis (SSPE), lead encephalopathy, tuberous sclerosis, Pantothenate kinase-associated neurodegeneration, and lipofuscinosis.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “operably linked” refers to a juxtaposition of described components wherein the components are in a relationship permitting them to function in their intended manner. A control sequence “operably linked” to a coding sequence is ligated in such a way that expression of the coding sequence is achieved under conditions compatible with the control sequences. “Operably linked” sequences may include both expression control sequences that are contiguous with the gene of interest and expression control sequences that act in trans or at a distance to control the gene of interest. The term “expression control sequence” refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (such as, a Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing and can also include additional components whose presence is advantageous, for example, leader sequences and fusion partner sequences. Unless stated otherwise, a description or statement herein of inserting a nucleic acid molecule encoding a fusion protein of the invention into an expression vector means that the inserted nucleic acid has also been operably linked within the vector to a functional promoter and other transcriptional and translational control elements required for expression of the encoded fusion protein when the expression vector containing the inserted nucleic acid molecule is introduced into compatible host cells or compatible cells of an organism.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The terms “disease” and “disorder” are used interchangeably to indicate a pathological state identified according to acceptable medical standards and practices in the art.

As used herein, the term “effective amount” refers to the amount of a therapy that is sufficient to reduce or ameliorate the severity and/or duration of a disease or one or more symptoms thereof; to prevent the advancement of a detrimental or pathological state; to cause regression of a pathological state; to prevent recurrence, development, onset, or progression of one or more symptoms associated with a pathological state; to detect a disorder; or to enhance or improve the prophylactic or therapeutic effect(s) of a therapy (e.g., the administration of another prophylactic or therapeutic agent).

As used herein, the term “J domain” refers to a fragment which retains the ability to accelerate the intrinsic ATPase catalytic activity of Hsp70 and its cognate. The J domains of a variety of J proteins have been determined (see, for example, Kampinga et al. (2010) Nat. Rev., 11:579-592; Hennessy et al. (2005) Protein Science, 14: 1697-1709, each of which is incorporated by reference in its entirety), and are characterized by a number of hallmarks: which is characterized by four α-helices (I, II, III, IV) and usually have the highly conserved tripeptide sequence motif of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif, which is necessary for stimulation of Hsp70 ATPase activity. As used herein, the term “J domain” is meant to include natural J domain sequences and functional variants thereof which retain the ability to accelerate Hsp70 intrinsic ATPase activity, which can be measured using methods well known in the art (see, for example, Horne et al. (2010) J. Biol. Chem., 285, 21679-21688, which is incorporated herein by reference in its entirety). A non-limiting list of human J domains is provided in Table 1.

### DETAILED DESCRIPTION

The present inventors have found that contacting certain cells with a fusion protein construct comprising a J domain of a J protein and a tau-binding domain have the unexpected effect of reducing the hyperphosphorylated Tau proteins. Hyperphosphorylated tau proteins are believed to cause a number of devastating diseases, including, but not limited to, Alzheimer's Disease (AD), Parkinson's Disease (PD), Primary age-related tauopathy (PART), Chronic traumatic encephalopathy (CTE), Progressive supranuclear palsy (PSP), Corticobasal degeneration (CBD), Frontotemporal dementia and parkinsonism linked to chromosome 17 (FTDP-17), Lytico-bodig disease, Ganglioglioma and gangliocytoma, Meningioangiomatosis, Postencephalitic parkinsonism, Subacute sclerosing panencephalitis (SSPE), lead encephalopathy, tuberous sclerosis, Pantothenate kinase-associated neurodegeneration, and lipofuscinosis. Accordingly, useful compositions and methods to treat tau-related disorders, e.g., in a subject in need thereof, are provided herein.

To overcome issues associated with chaperone-based therapies, we investigated whether it would be possible to design artificial chaperone proteins with high specificity. We designed a series of fusion protein constructs comprising an effector domain for Hsp70 binding/activation (J domain sequence), and a domain conferring specificity to tau proteins. The resulting fusion proteins act to accelerate the intrinsic ATPase catalytic activity of Hsp70 and its cognate, resulting in increased protein folding, reduced aggregation and/or accelerated clearance.

I. Fusion Protein Constructs

a. J Domains Useful in the Invention

J domains of a variety of J proteins have been determined. See, for example, Kampinga et al., Nat. Rev., 11:579-592 (2010); Hennessy et al., Protein Science, 14:1697-1709 (2005). A J domain useful in preparing a fusion protein of the invention has the key defining features of a J domain which principally accelerates HSP70 ATPase activity. Accordingly, an isolated J domain useful in the invention comprises a polypeptide domain, which is characterized by four α-helices (I, II, III, IV) and usually have the highly conserved tripeptide sequence of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is fundamental to primitive activity. Representative J domains include, but are not limited, a J domain of a DnaJB1, DnaJB2, DnaJB6, DnaJC6, a J domain of a large T antigen of SV40, and a J domain of a mammalian cysteine string protein (CSP-α). The amino acid sequences for these and other J domains that may be used in fusion proteins of the invention are provided in Table 1. The conserved HPD motif is highlighted in bold.

TABLE 1
Representative Human J Domain Sequences

| Protein | SEQ ID | Gene NCBI | Protein NCBI | J domain amino |
|---|---|---|---|---|
| Name | NO: | Reference | Reference | acid sequence |
| DNAJA1 | 1 | NM_001539 | NP_001530 | TYYDVLGVKPNATQEELKKAYRKL |
| | | | | ALKYHPDKNPNEGEKFKQISQAYE |
| | | | | VLSDAKKRELYDKGG |
| DNAJA2 | 2 | NM_005880 | NP_005871 | KLYDILGVPPGASENELKKAYRKL |
| | | | | AKEYHPDKNPNAGDKFKEISFAYE |
| | | | | VLSNPEKRELYDRYG |
| DNAJA3 | 3 | NM_001135110 | NP_001128582 | DYYQILGVPRNASQKEIKKAYYQL |
| | | | | AKKYHPDTNKDDPKAKEKFSQLAE |
| | | | | AYEVLSDEVKRKQYDAYG |
| DNAJA4 | 4 | NM_001130182 | NP_001123654 | ETQYYDILGVKPSASPEEIKKAYR |
| | | | | KLALKYHPDKNPDEGEKFKLISQA |
| | | | | YEVLSDPKKRDVYDQGGEQ |
| DNAJB1 | 5 | | | GKDYYQTLGLARGASDEEIKRAYR |
| | | | | RQALRYHPDKNKEPGAEEKFKEIA |
| | | | | EAYDVLSDPRKREIFDRYGEE |
| DNAJB2 | 6 | NM_001039550 | NP_001034639 | ASYYEILDVPRSASADDIKKAYRR |
| | | | | KALQWHPDKNPDNKEFAEKKFKEV |
| | | | | AEAYEVLSDKHKREIYDRYGRE |
| DNAJB3 | 7 | NM_001001394 | NP_001001394 | MVDYYEVLDVPRQASSEAIKKAYR |
| | | | | KLALKWHPDKNPENKEEAERRFKQ |
| | | | | VAEAYEVLSDAKKRDIYDRYG |
| DNAJB4 | 8 | NM_001317099 | NP_001304028 | GKDYYCILGIEKGASDEDIKKAYR |
| | | | | KQALKFHPDKNKSPQAEEKFKEVA |
| | | | | EAYEVLSDPKKREIYDQFGEE |
| DNAJB5 | 9 | NM_001135004 | NP_001128476 | DYYKILGIPSGANEDEIKKAYRKM |
| | | | | ALKYHPDKNKEPNAEEKFKEIAEA |
| | | | | YDVLSDPKKRGLYDQYG |
| DNAJB6 | 10 | NM_005494 | NP_005485 | VDYYEVLGVQRHASPEDIKKAYRK |
| | | | | LALKWHPDKNPENKEEAERKFKQV |
| | | | | AEAYEVLSDAKKRDIYDKYG |
| DNAJB7 | 11 | NM_145174 | NP_660157 | DYYEVLGLORYASPEDIKKAYHKV |
| | | | | ALKWHPDKNPENKEEAERKEKEVA |
| | | | | EAYEVLSNDEKRDIYDKYG |
| DNAJB8 | 12 | NM_153330 | NP_699161 | NYYEVLGVQASASPEDIKKAYRKL |
| | | | | ALRWHPDKNPDNKEEAEKKFKLVS |
| | | | | EAYEVLSDSKKRSLYDRAG |
| DNAJB9 | 13 | NM_012328 | NP_036460 | SYYDILGVPKSASERQIKKAFHKL |
| | | | | AMKYHPDKNKSPDAEAKFREIAEA |
| | | | | YETLSDANRRKEYDTLG |
| DNAJB11 | 14 | NM_016306 | NP_057390 | DFYKILGVPRSASIKDIKKAYRKL |
| | | | | ALQLHPDRNPDDPQAQEKFQDLGA |
| | | | | AYEVLSDSEKRKQYDTYG |
| DNAJB12 | 15 | NM_001002762 | NP_001002762 | YEILGVSRGASDEDLKKAYRRLAL |
| | | | | KFHPDKNHAPGATEAFKAIGTAYA |
| | | | | VLSNPEKRKQYDQFGDD |
| DNAJB13 | 16 | NM_153614 | NP_705842 | DYYSVLGITRNSEDAQIKQAYRRL |
| | | | | ALKHHPLKSNEPSSAEIFRQIAEA |
| | | | | YDVLSDPMKRGIYDKFG |
| DNAJB14 | 17 | NM_001031723 | NP_001026893 | NYYEVLGVTKDAGDEDLKKAYRKL |
| | | | | ALKFHPDKNHAPGATDAFKKIGNA |
| | | | | YAVLSNPEKRKQYDLTG |
| DNAJC1 | 18 | NM_022365 | NP_071760 | NFYQFLGVQQDASSADIRKAYRKL |
| | | | | SLTLHPDKNKDENAETQFRQLVAI |
| | | | | YEVLKDDERRQRYDDIL |
| DNAJC2 | 19 | NM_001129887 | NP_001123359 | DHYAVLGLGHVRYKATQRQIKAAH |
| | | | | KAMVLKHHPDKRKAAGEPIKEGDN |
| | | | | DYFTCITKAYEMLSDPVKRRAFNS |
| | | | | VD |
| DNAJC3 | 20 | NM_006260 | NP_006251 | DYYKILGVKRNAKKQEIIKAYRKL |
| | | | | ALQWHPDNFQNEEEKKKAEKKFID |
| | | | | IAAAKEVLSDPEMRKKEDDGE |
| DNAJC4 | 21 | NM_001307980 | NP_001294909 | TYYELLGVHPGASTEEVKRAFFSK |
| | | | | SKELHPDRDPGNPSLHSRFVELSE |
| | | | | AYRVLSREQSRRSYDDQL |
| DNAJC5 | 22 | NM_025219 | NP_079495 | GESLYHVLGLDKNATSDDIKKSYR |
| | | | | KLALKYHPDKNPDNPEAADKFKEI |
| | | | | NNAHAILTDATKRNIYDKYGSL |
| DNAJC5B | 23 | NM_033105 | NP_149096 | ALYEILGLHKGASNEEIKKTYRKL |
| | | | | ALKHHPDKNPDDPAATEKFKEINN |
| | | | | AHAILTDISKRSIYDKYG |
| DNAJC6 | 24 | NM_001256864 | NP_001243793 | TKWKPVGMADLVTPEQVKKVYRKA |
| | | | | VLVVHPDKATGQPYEQYAKMIFME |
| | | | | LNDAWSEFENQGQKPLY |
| DNAJC7 | 25 | NM_001144766 | NP_001138238 | DYYKILGVDKNASEDEIKKAYRKR |
| | | | | ALMHHPDRHSGASAEVOKEEEKKF |
| | | | | KEVGEAFTILSDPKKKTRYDSGQ |
| DNAJC8 | 26 | NM_014280 | NP_055095 | NPFEVLQIDPEVTDEEIKKRFRQL |
| | | | | SILVHPDKNQDDADRAQKAFEAVD |
| | | | | KAYKLLLDQEQKKRALDVIQ |
| DNAJC9 | 27 | NM_015190 | NP_056005 | DLYRVLGVRREASDGEVRRGYHKV |
| | | | | SLQVHPDRVGEGDKEDATRRFQIL |
| | | | | GKVYSVLSDREQRAVYDEQG |
| DNAJC10 | 28 | NM_001271581 | NP_001258510 | DFYSLLGVSKTASSREIRQAFKKL |
| | | | | ALKLHPDKNPNNPNAHGDELKINR |
| | | | | AYEVLKDEDLRKKYDKYG |
| DNAJC11 | 29 | NM_018198 | NP_060668 | DYYSLLNVRREASSEELKAAYRRL |
| | | | | CMLYHPDKHRDPELKSQAERLENL |
| | | | | VHQAYEVLSDPOTRAIYDIYG |
| DNAJC12 | 30 | NM_021800 | NP_068572 | DYYTLLGCDELSSVEQILAEFKVR |
| | | | | ALECHPDKHPENPKAVETFQKLOK |
| | | | | AKEILTNEESRARYDHWR |
| DNAJC13 | 31 | NM_015268 | NP_056083 | DAYEVLNLPQGQGPHDESKIRKAY |
| | | | | FRLAQKYHPDKNPEGRDMFEKVNK |
| | | | | AYEFLCTKSAKIVDGPDP |
| DNAJC14 | 32 | NM_032364 | NP_115740 | NPFHVLGVEATASDVELKKAYRQL |
| | | | | AVMVHPDKNHHPRAEEAFKVLRAA |
| | | | | WDIVSNAEKRKEYEMKR |
| DNAJC15 | 33 | NM_013238 | NP_037370 | EAGLILGVSPSAGKAKIRTAHRRV |
| | | | | MILNHPDKGGSPYVAAKINEAKDL |
| | | | | LETTTKH |
| DNAJC16 | 34 | | | DPYRVLGVSRTASQADIKKAYKKL |
| | | | | AREWHPDKNKDPGAEDKFIQISKA |
| | | | | YEILSNEEKRSNYDQYG |
| DNAJC17 | 35 | NM_018163 | NP_060633 | DLYALLGIEEKAADKEVKKAYRQK |
| | | | | ALSCHPDKNPDNPRAAELFHOLSQ |
| | | | | ALEVLTDAAARAAYDKVR |
| DNAJC18 | 36 | NM_152686 | NP_689899 | NYYEILGVSRDASDEELKKAYRKL |
| | | | | ALKFHPDKNCAPGATDAFKAIGNA |
| | | | | FAVLSNPDKRLRYDEYG |
| DNAJC19 | 37 | NM_001190233 | NP_001177162 | EAALILGVSPTANKGKIRDAHRRI |
| | | | | MLLNHPDKGGSPYIAAKINEAKDL |
| | | | | LEGQAKK |
| DNAJC20 | 38 | NM_172002 | NP_741999 | DYFSLMDCNRSFRVDTAKLQHRYQ |
| | | | | QLQRLVHPDFFSQRSQTEKDESEK |
| | | | | HSTLVNDAYKTLLAPLSRGLYLLK |
| DNAJC21 | 39 | NM_001012339 | NP_001012339 | CHYEALGVRRDASEEELKKAYRKL |
| | | | | ALKWHPDKNLDNAAEAAEQFKLIQ |
| | | | | AAYDVLSDPQERAWYDNHR |
| DNAJC22 | 40 | NM_001304944 | NP_001291873 | LAYQVLGLSEGATNEEIHRSYQEL |
| | | | | VKVWHPDHNLDQTEEAQRHFLEIQ |
| | | | | AAYEVLSQPRKPWGSRR |
| DNAJC23 | 41 | NM_007214 | NP_009145 | NPYEVLNLDPGATVAEIKKQYRLL |
| | | | | SLKYHPDKGGDEVMFMRIAKAYAA |
| | | | | LTDEESRKNWEEFG |
| DNAJC24 | 42 | NM_181706 | NP_859057 | DWYSILGADPSANISDLKQKYQKL |
| | | | | ILMYHPDKOSTDVPAGTVEECVOK |
| | | | | FIEIDQAWKILGNEETKREYDLQR |
| DNAJC25 | 43 | NM_001015882 | NP_001015882 | DCYEVLGVSRSAGKAEIARAYROL |
| | | | | ARRYHPDRYRPQPGDEGPGRTPQS |
| | | | | AEEAFLLVATAYETLKDEETRKDY |
| | | | | DYML |
| DNAJC26 | 44 | NM_001318134 | NP_001305063 | SRWTPVGMADLVAPEQVKKHYRRA |
| | | | | VLAVHPDKAAGQPYEQHAKMIFME |
| | | | | LNDAWSEFENQGSRPLF |
| DNAJC27 | 45 | | | DSWDMLGVKPGASRDEVNKAYRKL |
| | | | | AVLLHPDKCVAPGSEDAFKAVVNA |
| | | | | RTALLKNIK |
| DNAJC28 | 46 | NM_001040192 | NP_001035282 | EYYRLLNVEEGCSADEVRESFHKL |
| | | | | AKQYHPDSGSNTADSATFIRIEKA |
| | | | | YRKVLSHVIEQTNASQS |
| DNAJC29 | 47 | NM_014363 | NP_055178 | ILKEVTSVVEQAWKLPESERKKII |
| | | | | RRLYLKWHPDKNPENHDIANEVFK |
| | | | | HLQNEINRLEKQAFLDQNADRASR |
| | | | | RTFSTSASRFQSDKYS |
| DNAJC30 | 48 | NM_032317 | NP_115693 | ALYDLLGVPSTATQAQIKAAYYRQ |
| | | | | CFLYHPDRNSGSAEAAERFTRISQ |
| | | | | AYVVLGSATLRRKYDRGL |

b. Tau-Binding Domain

The fusion protein also comprises at least one Tau-binding domain. The tau-binding domain can be a single chain polypeptide, or a multimeric polypeptide joined with the J domain to form the fusion protein.

It is ideal that the tau-binding domain possesses a sufficient affinity to be able to bind the tau protein when present at a pathological level within cells. Therefore, in one embodiment, the fusion protein comprises a tau-binding domain that has a K_{D} for tau of, for example, 2 μM or less, 1 μM or less, 500 nM or less, 300 nM or less, 100 nM or less, 30 nM or less when tested by ELISA on 96 well microtiter plates. In another embodiment, the fusion protein comprises a tau-binding domain that has a K_{D} for the aggregated form of tau of, for example, 2 μM or less, 1 μM or less, 500 nM or less, 300 nM or less, 100 nM or less, 30 nM or less when tested by ELISA on 96 well microtiter plates. In still another embodiment, the tau-binding domain has selectivity for the aggregated form of tau; for example, the tau-binding domain has at least two-fold higher, at least 3 fold higher, at least 4 fold higher, at least 5 fold higher, at least 10 fold higher, at least 30 fold higher, at least 100 fold higher affinity for the aggregated form of tau when compared with the affinity for the soluble form of tau.

Tau-binding domains have been previously identified and characterized (see, for example, U.S. Pat. No. 7,605,133, WO 2019/161386, Abe et al., (2007) BMC Bioinformatics, 8:451, each of which is incorporated herein by reference). Therefore, in another embodiment, the fusion protein comprises a tau-binding domain that is selected from the group consisting of SEQ ID NOs: 49-52 (see, for example, Table 2). In one particular embodiment, the fusion protein comprises the tau-binding domain of SEQ ID NO: 49.

In another embodiment, the fusion protein also contemplates the use of the tau-binding domain that is chemically conjugated to the J domain. The tau-binding domain can be conjugated directly to the J domain. Alternatively, it can be conjugated to the J domain by a linker. For example, there are a large number of chemical cross-linking agents that are known to those skilled in the art and useful for cross-linking the tau-binding domain to the J domain, or a targeting domain to a fusion protein comprising the tau-binding domain and J domain. For example, some cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl(4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), succinimidyl 6-[3-(2-pyridyldithio)propionate]hexanoate (LC-SPDP). Cross-linking agents having N-hydroxysuccinimide moieties can be obtained as the N-hydroxysulfosuccinimide analogs, which generally have greater water solubility. In addition, cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al., (1990) Bioconj. Chem. 1:2-12, incorporated by reference herein.

TABLE 2
Examples of tau-binding domains

| | SEQ ID | | |
|---|---|---|---|
| Name | NO: | Length | Sequence |
| TBP1 | 49 | 6 | VQIVYK |
| scFv(Tau) | 50 | 234 | EVQLQQSGAELVQPGASVKLSCTASGFNIKDTSMHWVRQ |
| | | | RPEQGLEWIGRIAPANGNTKYDPKFQGKATITTDTSSNT |
| | | | AYLQLSSLTSEDTAVYYCSGSGNYDWGQGTTLTVSGGGG |
| | | | SGGGGSGGGGSDIQMNQSPSSLSASLGDTITISCHASQN |
| | | | INVWLSWYQQKPGNIPKLLIYEASTLYTGVPSRFSGSGS |
| | | | GTGFTLTISSLOPEDIATYYCQQGQSYPWTFGGGTKLEI |
| scFv(MW7) | 51 | 243 | QVKLQESGGGLVQPGGSMKLSCAASGFTFSDAWMDWVRQ |
| | | | SPEKGLSGVAEIRSKANNHATYYAESVKGRFTISRDDSK |
| | | | SSVYLOMNSLRAEDTGIYYCIYAGFAYWGQGTTVTVSSG |
| | | | GGGSGGGGSGGGGSDIELTQSPSSLAMSVGQKVTMSCKS |
| | | | SQSLLNSSNQKNYLAWYQQKPGOSPKLLVYFASTRESGV |
| | | | PDRFIGSGSGTDFTLTISSVQAEDLADYFCQQHYSTPWT |
| | | | FGGGTKLEI |
| Happ1 | 52 | 118 | MQSVLTQPPSASGTPGQRVTISCSGSSSNIGSNYVYWYQ |
| | | | QLPGTAPKLLIYRNNQRPSGVPDRFSGSKSGTSASLAIS |
| | | | GLRPEDEADYYCAAWDDSLCVALVEGGGTNGGGGVDGTA |
| | | | G |
| TBP2 | 53 | 6 | VQIINK |
| TBP3 | 54 | 13 | YQQYQDATADEQG |

c. Optional Linker

The fusion proteins described herein can optionally contain one or more linkers. Linkers can be peptidic or non-peptidic. The purpose of the linker is to provide, among other things, an adequate distance between functional domains within the protein (e.g., between the J domain and tau-binding domain, between tandem arrangements of tau-binding domains, between either the J domain and tau-binding domain and an optional targeting reagent, or between either the J domain and tau-binding domain and an optional detection domain or epitope) for optimal function of each of the domains. Clearly, a linker preferably does not interfere with the respective functions of the J domain, the target protein binding domain of a fusion protein according to the invention. A linker, if present in a fusion protein of the invention, is selected to attenuate the cytotoxicity caused by target proteins (tau proteins), and it may be omitted if direct attachment achieves a desired effect. Linkers present in a fusion protein of the invention may comprise one or more amino acids encoded by a nucleotide sequence present on a segment of nucleic acid in or around a cloning site of an expression vector into which is inserted in frame a nucleic acid segment encoding a protein domain or an entire fusion protein as described herein. In one embodiment, the peptide linker is between 1 amino acid and 20 amino acids in length. In another embodiment, the peptide linker is between 2 amino acids and 15 amino acids in length. In still another embodiment, the peptide linker is between 2 amino acids and 10 amino acids in length.

Selecting one or more polypeptide linkers to produce a fusion protein according to the invention is within the knowledge and skill of practitioners in the art. See, for example, Arai et al., Protein Eng., 14(8): 529-532 (2001); Crasto et al., Protein Eng., 13(5): 309-314 (2000); George et al., Protein Eng., 15(11): 871-879 (2003); Robinson et al., Proc. Natl. Acad. Sci. USA, 95: 5929-5934 (1998), each of which is incorporated herein by reference in its entirety. Examples of linkers of two or more amino acids that may be used in preparing a fusion protein according to the invention, include, by are not limited to, those provided below in Table 3.

TABLE 3
Linker Sequences

| SEQ ID NO: | Length | Sequence |
|---|---|---|
| 55 | 2 | SR |
| 56 | 4 | GTGS |
| 57 | 5 | GLESR |
| 58 | 4 | GGSG |
| 59 | 4 | GGGS |
| 60 | 5 | DIAAA |
| 61 | 9 | DIAAALESR |
| 62 | 15 | GGGGSGGGGSGGGGS |
| 63 | 11 | AEAAAKEAAAK |
| 64 | 15 | SGGGSGGGGSGGGGS |
| 65 | 25 | DIGGGGSGGGGGGGGSGGGGSAAA |

d. Targeting Reagents

The fusion proteins disclosed herein can further comprise a targeting moiety. As used herein, the terms “targeting moiety” and “targeting reagent” are used interchangeably and refer to a substance associated with the fusion protein that enhances binding, transport, accumulation, residence time, bioavailability, or modifies biological activity or therapeutic effect of the fusion protein in a cell or in the body of a subject. A targeting moiety can have functionality at the tissue, cellular, and/or subcellular level. The targeting moiety can direct localization of the fusion protein to a particular cell, tissue or organ, for example, upon administration of the fusion protein into a subject. In one embodiment, the targeting moiety is located at the N-terminus of the fusion protein. In another embodiment, the targeting moiety is located at the C-terminus of the fusion protein. In still another embodiment, the targeting moiety is located between the N-terminus and C-terminus of the fusion protein. In another embodiment, the targeting moiety is attached to the fusion protein via chemical conjugation.

The targeting moiety can include, but is not limited to, an organic or inorganic molecule, a peptide, a peptide mimetic, a protein, an antibody or fragment thereof, a growth factor, an enzyme, a lectin, an antigen or immunogen, viruses or component thereof, a viral vector, a receptors, a receptor ligand, a toxin, a polynucleotide, an oligonucleotide or aptamer, a nucleotide, a carbohydrate, a sugar, a lipid, a glycolipid, a nucleoprotein, a glycoprotein, a lipoprotein, a steroid, a hormone, a chemoattractant, a cytokine, a chemokine, a drug, or a small molecule, among others.

In an exemplary embodiment of the present invention, the targeting moiety enhances binding, transport, accumulation, residence time, bioavailability, or modifies biological activity or therapeutic effect of the platform, or its associated ligand and/or active agent in the target cell or tissue, for example, neuronal cells of the CNS, and/or the PNS. Thus, the targeting moiety can have specificity for cellular receptors associated with the CNS, or is otherwise associated with enhanced delivery to the CNS via the blood-brain barrier (BBB). Consequently, a ligand, as described above, can be both a ligand and a targeting moiety.

In some embodiments, the targeting moiety can be a cell-penetrating peptide, for example, as described in U.S. Pat. No. 10,111,965, which is incorporated by reference in its entirety. In another embodiment, the targeting moiety can be an antibody or an antigen-binding fragment or single-chain derivative thereof, for example, as described in U.S. Ser. No. 16/131,591, which is incorporated herein by reference in its entirety.

The targeting moiety can be coupled to the platform for targeted cellular delivery by being directly or indirectly bound to the core. For example, in embodiments where the core comprises a nanoparticle, conjugation of the targeting moiety to the nanoparticle can utilize similar functional groups that are employed to tether PEG to the nanoparticle. Thus, the targeting moiety can be directly bound to the nanoparticle through functionalization of the targeting moiety. Alternatively, the targeting moiety can be indirectly bound to the nanoparticle through conjugation of the targeting moiety to a functionalized PEG, as discussed above. A targeting moiety can be attached to the core by way of covalent, non-covalent, or electrostatic interactions. In one embodiment, the targeting moiety is a peptide. In a particular embodiment, the targeting moiety is a peptide that is covalently attached to the N-terminus of the fusion protein.

e. Epitopes

In certain embodiments, the fusion protein of the present invention contains an optional epitope or tag, which can impart additional properties to the fusion protein. As used herein, the terms “epitope” and “tag” are used interchangeably to refer to an amino acid sequence, typically 300 amino acids or less in length, which is typically attached to the N-terminal or C-terminal end of the fusion protein. In one embodiment, the fusion protein of the present invention further comprises an epitope which is used to facilitate purification. Examples of such epitopes useful for purification, provided below in Table 4, include the human IgG1 Fc sequence (SEQ ID NO: 66), the FLAG epitope (DYKDDDDK, SEQ ID NO: 67), His6 epitope (SEQ ID NO: 68), c-myc (SEQ ID NO: 69), HA (SEQ ID NO: 70), V5 epitope (SEQ ID NO: 71), or glutathione-s-transferase (SEQ ID NO: 72). In another embodiment, the fusion protein of the present invention further comprises an epitope which is used to increase the half-life of the fusion protein when administered into a subject, for example a human. Examples of such epitopes useful for increasing half-life include the human Fc sequence. Therefore, in one particular embodiment, the fusion protein comprises, in addition to a J domain and tau-binding domain, a human Fc epitope. The epitope is positioned at the C-terminal end of the fusion protein.

TABLE 4
Representative Examples of Epitopes

| SEQ ID | | | |
|---|---|---|---|
| NO: | EPITOPE | LENGTH | SEQUENCE |
| 66 | Human IgG1 | 232 | EPKSCDKTHTCPPCPAPELLGGPSVFLFPPKPKDTLMISRTP |
| | Fc domain | | EVTCVVVDVSHEDPEVKFNWYVDGVEVHNAKTKPREEQYNST |
| | | | YRVVSVLTVLHQDWLNGKEYKCKVSNKALPAPIEKTISKAKG |
| | | | QPREPQVYTLPPSRDELTKNQVSLTCLVKGFYPSDIAVEWES |
| | | | NGQPENNYKTTPPVLDSDGSFFLYSKLTVDKSRWQQGNVFSC |
| | | | SVMHEALHNHYTQKSLSLSPGK |
| 67 | FLAG epitope | 8 | DYKDDDDK |
| 68 | His6 | 6 | HHHHHH |
| 69 | c-myc | 10 | EQKLISEEDL |
| 70 | HA | 9 | YPYDVPDYA |
| 71 | V5 epitope | 14 | GKPIPNPLLGLDST |
| 72 | Glutathione- | 220 | MSPILGYWKIKGLVQPTRLLLEYLEEKYEEHLYERDEGDKWR |
| | S-transferase | | NKKFELGLEFPNLPYYIDGDVKLTQSMAIIRYIADKHNMLGG |
| | | | CPKERAEISMLEGAVLDIRYGVSRIAYSKDFETLKVDFLSKL |
| | | | PEMLKMFEDRLCHKTYLNGDHVTHPDFMLYDALDVVLYMDPM |
| | | | CLDAFPKLVCFKKRIEAIPQIDKYLKSSKYIAWPLQGWQATF |
| | | | GGGDHPPKSD |

f. Cell-Penetrating Peptides

In still other embodiments, the fusion protein described herein can further comprise a cell-penetrating peptide. Cell-penetrating peptides are known to carry a conjugated cargo, whether a small molecule, peptide, protein or nucleic acid, into cells. Non-limiting examples of cell-penetrating peptides in a fusion protein of the invention include, but are not limited to, a polycationic peptide, e.g., an HIV TAT peptide49-57, polyarginines, and penetratin pAntan(43-58), amphipathic peptide, e.g., pep-1, a hydrophobic peptide, e.g., a C405Y, and the like. See Table 5 below.

TABLE 5
Examples of Cell-Penetrating Peptides

| SEQ ID NO: | SEQUENCE |
|---|---|
| 73 | RKKRRQRRR |
| 74 | RQIKWFQNRRMKWKK |
| 75 | KETWWETWWTEWSQPKKKRKV |
| 76 | CSIPPEVKFNKPFVYLI |

Therefore, in one embodiment, the fusion protein comprises a cell-penetrating peptide and a fusion protein, wherein the cell-penetrating peptide is selected from the group consisting of SEQ ID NOs: 73-76, and the fusion protein is selected from the group consisting of SEQ ID NOs: 83-88 and 95-101. In another embodiment, the fusion protein comprises the cell-penetrating peptide of SEQ ID NO: 73, and the fusion protein selected from the group consisting of SEQ ID NOs: 83-88 and 95-101. In another embodiment, the fusion protein comprises the cell-penetrating peptide of SEQ ID NO: 74, and the fusion protein selected from the group consisting of SEQ ID NOs: 83-88 and 95-101. In still another embodiment, the fusion protein comprises the cell-penetrating peptide of SEQ ID NO: 75, and the fusion protein selected from the group consisting of SEQ ID NOs: 83-88 and 95-101. In yet another embodiment, the fusion protein comprises the cell-penetrating peptide of SEQ ID NO: 76, and the fusion protein selected from the group consisting of SEQ ID NOs: 83-88 and 95-101. Cells expressing the fusion protein constructs with the cell-penetrating peptide can be administered to a subject, for example a human subject (e.g., a patient having or at risk of suffering from a tau disorder). The fusion protein is secreted from the cells, which help reduce tau-containing protein aggregation and/or associated cytotoxicity.

g. Arrangement of J Domain and Tau Binding Domain

The fusion proteins described herein can be arranged in a multitude of ways. In one embodiment, the tau-binding domains attached to the C-terminal side of the J domain. In another embodiment, the tau-binding domains attached to the N-terminal side of the J domain. The tau-binding domain and the J domain, in either configuration, can optionally be separated via a linker as described above.

In some embodiments, the J domain can be attached to a plurality of tau-binding domains, for example, two tau-binding domains, three tau-binding domains, four tau-binding domains or more. The plurality of tau-binding domains can be attached to the N-terminal side of the J domain. In still another embodiment, the plurality tau-binding domains can be attached on the N-terminal and C-terminal sides of the J domain. Each of the plurality of tau-binding domains can be the same tau-binding domain. In another embodiment, each of the plurality of tau-binding domains in the fusion protein can be different tau-binding domains (i.e., different sequences).

In some embodiments, the fusion proteins can comprise a structure selected from the following group:

| | |
|---|---|
| a. | DNAJ-X-T |
| b. | DNAJ-X-T-X-T |
| c. | DNAJ-X-T-X-T-X-T |
| d. | T-X-DNAJ |
| e. | T-X-T-X-DNAJ |
| f. | T-X-T-X-T-X-DNAJ |
| g. | T-X-DNAJ-X-T, |
| h. | T-X-DNAJ-X-T-X-T, |
| i. | T-X-DNAJ-X-T-X-T-X-T, |
| j. | T-X-T-X-DNAJ-X-T |
| k. | T-X-T-X-DNAJ-X-T-X-T, |
| l. | T-X-T-X-DNAJ-X-T-X-T-X-T, |
| m. | T-X-T-X-T-X-DNAJ-X-T, |
| n. | T-X-T-X-T-X-DNAJ-X-T-X-T, and |
| o. | T-X-T-X-T-X-DNAJ-X-T-X-T-X-T, |

wherein,
T is a tau-binding domain,
DNAJ is a J domain of a J protein, and
X is an optional linker.

In one embodiment, the fusion protein comprises the J domain selected from the group consisting of SEQ ID NOs: 5, 6, 10, 24, and 31. In one particular embodiment, the fusion protein comprises the J domain of SEQ ID NO: 5.

In another embodiment, the tau-binding domain is selected from the group consisting of SEQ ID NOs: 49-54. In one particular embodiment, the tau-binding domain is SEQ ID NO:49.

In still another embodiment, the fusion protein comprises the J domain of SEQ ID NO: 5, and the tau-binding domain of SEQ ID NO: 49. In another embodiment, the fusion protein comprises the J domain of SEQ ID NO: 5, and at least two copies of the tau-binding domain of SEQ ID NO: 49. In another embodiment, the fusion protein comprises the J domain of SEQ ID NO: 5, and the tau-binding domain of SEQ ID NO: 50. In another embodiment, the fusion protein comprises the J domain of SEQ ID NO: 5, and the tau-binding domain of SEQ ID NO: 51. In yet another embodiment, the fusion protein comprises the J domain of SEQ ID NO: 5, and the tau-binding domain of SEQ ID NO: 52. In another embodiment, the fusion protein comprises the J domain of SEQ ID NO: 5, and the tau-binding domain of SEQ ID NO: 53. In yet another embodiment, the fusion protein comprises the J domain of SEQ ID NO: 5, and the tau-binding domain of SEQ ID NO: 54.

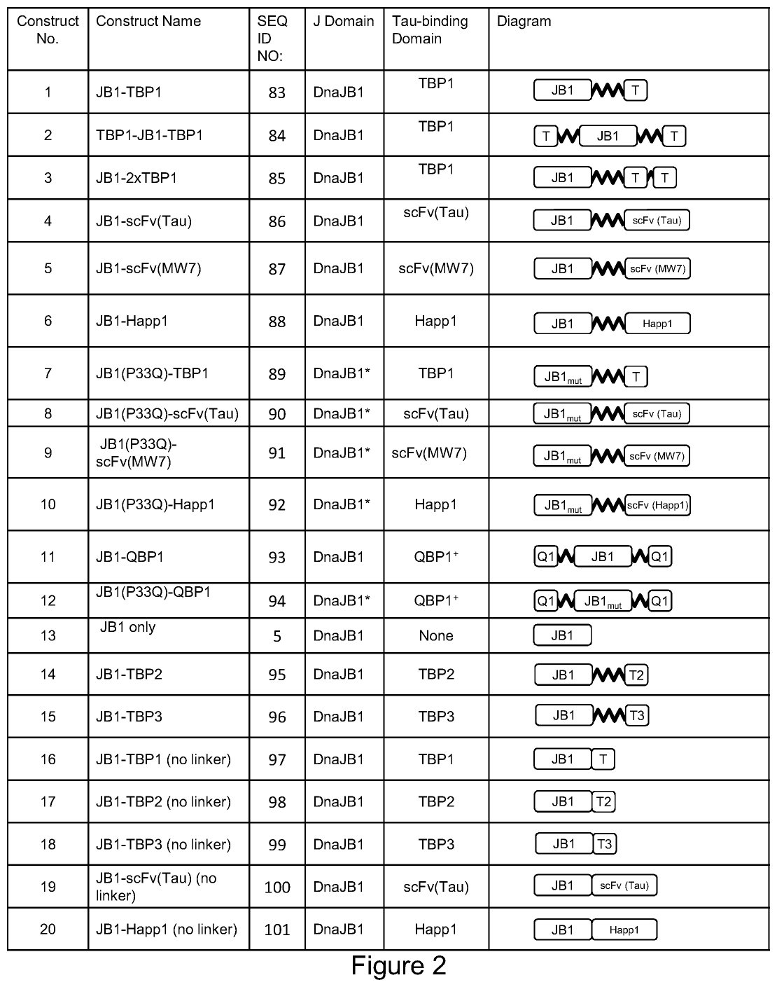

Non-limiting examples of fusion protein constructs comprising a J domain and tau-binding domain are depicted schematically in FIG. 2, and also shown below in Table 6. In another embodiment, the specific fusion protein construct is selected from the group consisting of SEQ ID NOs: 83-88 and 95-101.

TABLE 6
Fusion Protein Constructs and Control Constructs

| Construct | SEQ ID | Construct | | |
|---|---|---|---|---|
| No | NO: | Name | Length | Sequence |
| 1 | 83 | JB1-TBP1 | 106 | MGKDYYQTLGLARGASDEEIKRAYRRQALRYHP |
| | | | | DKNKEPGAEEKFKEIAEAYDVLSDPRKREIFDR |
| | | | | YGEEGLKGSDIGGGGSGGGGSGGGGSGGGGSAA |
| | | | | AVQIVYK |
| 2 | 84 | TBP1-JB1- | 147 | MGKPIPNPLLGLDSTGTGSVQIVYKGGGSGGGS |
| | | TBP1 | | GGGSGGGSMGKDYYQTLGLARGASDEEIKRAYR |
| | | | | RQALRYHPDKNKEPGAEEKFKEIAEAYDVLSDP |
| | | | | RKREIFDRYGEEGLKGSDIGGGGSGGGGSGGGG |
| | | | | SGGGGSAAAVQIVYK |
| 3 | 85 | JB1- | 128 | MGKDYYQTLGLARGASDEEIKRAYRRQALRYHP |
| | | 2XTBP1 | | DKNKEPGAEEKFKEIAEAYDVLSDPRKREIFDR |
| | | | | YGEEGLKGSDIGGGGSGGGGSGGGGSGGGGSAA |
| | | | | AVQIVYKGGGSGGGSGGGSGGGSVQIVYK |
| 4 | 86 | JB1- | 334 | MGKDYYQTLGLARGASDEEIKRAYRROALRYHP |
| | | scFv(Tau) | | DKNKEPGAEEKFKEIAEAYDVLSDPRKREIFDR |
| | | | | YGEEGLKGSDIGGGGSGGGGSGGGGSGGGGSAA |
| | | | | AEVOLQQSGAELVQPGASVKLSCTASGENIKDT |
| | | | | SMHWVRQRPEQGLEWIGRIAPANGNTKYDPKFQ |
| | | | | GKATITTDTSSNTAYLQLSSLTSEDTAVYYCSG |
| | | | | SGNYDWGQGTTLTVSGGGGSGGGGSGGGGSDIQ |
| | | | | MNQSPSSLSASLGDTITISCHASQNINVWLSWY |
| | | | | QQKPGNIPKLLIYEASTLYTGVPSRESGSGSGT |
| | | | | GFTLTISSLQPEDIATYYCQQGQSYPWTFGGGT |
| | | | | KLEI |
| 5 | 87 | JB1- | 343 | MGKDYYQTLGLARGASDEEIKRAYRRQALRYHP |
| | | scFv(MW7) | | DKNKEPGAEEKFKEIAEAYDVLSDPRKREIFDR |
| | | | | YGEEGLKGSDIGGGGSGGGGSGGGGSGGGGSAA |
| | | | | AQVKLQESGGGLVQPGGSMKLSCAASGFTFSDA |
| | | | | WMDWVRQSPEKGLSGVAEIRSKANNHATYYAES |
| | | | | VKGRFTISRDDSKSSVYLQMNSLRAEDTGIYYC |
| | | | | IYAGFAYWGQGTTVTVSSGGGGSGGGGSGGGGS |
| | | | | DIELTQSPSSLAMSVGQKVTMSCKSSQSLLNSS |
| | | | | NOKNYLAWYQQKPGQSPKLLVYFASTRESGVPD |
| | | | | RFIGSGSGTDFTLTISSVQAEDLADYFCQQHYS |
| | | | | TPWTFGGGTKLEI |
| 6 | 88 | JB1-Happ1 | 218 | MGKDYYQTLGLARGASDEEIKRAYRRQALRYHP |
| | | | | DKNKEPGAEEKFKEIAEAYDVLSDPRKREIFDR |
| | | | | YGEEGLKGSDIGGGGSGGGGSGGGGSGGGGSAA |
| | | | | AMQSVLTOPPSASGTPGQRVTISCSGSSSNIGS |
| | | | | NYVYWYQQLPGTAPKLLIYRNNQRPSGVPDRES |
| | | | | GSKSGTSASLAISGLRPEDEADYYCAAWDDSLC |
| | | | | VALVFGGGTNGGGGVDGTAG |
| 7 | 89 | JB1(P33Q)- | 106 | MGKDYYQTLGLARGASDEEIKRAYRRQALRYHQ |
| | | TBP | | DKNKEPGAEEKFKEIAEAYDVLSDPRKREIFDR |
| | | | | YGEEGLKGSDIGGGGSGGGGSGGGGSGGGGSAA |
| | | | | AVQIVYK |
| 8 | 90 | JB1(P33Q)- | 334 | MGKDYYQTLGLARGASDEEIKRAYRRQALRYHQ |
| | | scFv(Tau) | | DKNKEPGAEEKFKEIAEAYDVLSDPRKREIFDR |
| | | | | YGEEGLKGSDIGGGGSGGGGSGGGGSGGGGSAA |
| | | | | AEVOLQQSGAELVQPGASVKLSCTASGFNIKDT |
| | | | | SMHWVRORPEQGLEWIGRIAPANGNTKYDPKFQ |
| | | | | GKATITTDTSSNTAYLQLSSLTSEDTAVYYCSG |
| | | | | SGNYDWGQGTTLTVSGGGGSGGGGSGGGGSDIQ |
| | | | | MNQSPSSLSASLGDTITISCHASQNINVWLSWY |
| | | | | QQKPGNIPKLLIYEASTLYTGVPSRFSGSGSGT |
| | | | | GFTLTISSLOPEDIATYYCQQGQSYPWTFGGGT |
| | | | | KLEI |
| 9 | 91 | JB1(P33Q)- | 343 | MGKDYYQTLGLARGASDEEIKRAYRRQALRYHQ |
| | | scFv(MW7) | | DKNKEPGAEEKFKEIAEAYDVLSDPRKREIFDR |
| | | | | YGEEGLKGSDIGGGGSGGGGSGGGGSGGGGSAA |
| | | | | AQVKLQESGGGLVQPGGSMKLSCAASGFTFSDA |
| | | | | WMDWVRQSPEKGLSGVAEIRSKANNHATYYAES |
| | | | | VKGRFTISRDDSKSSVYLQMNSLRAEDTGIYYC |
| | | | | IYAGFAYWGQGTTVTVSSGGGGSGGGGSGGGGS |
| | | | | DIELTQSPSSLAMSVGQKVTMSCKSSQSLLNSS |
| | | | | NOKNYLAWYQQKPGQSPKLLVYFASTRESGVPD |
| | | | | RFIGSGSGTDFTLTISSVQAEDLADYFCQQHYS |
| | | | | TPWTFGGGTKLEI |
| 10 | 92 | JB1(P33Q)- | 218 | MGKDYYQTLGLARGASDEEIKRAYRRQALRYHQ |
| | | Happ1 | | DKNKEPGAEEKFKEIAEAYDVLSDPRKREIFDR |
| | | | | YGEEGLKGSDIGGGGSGGGGSGGGGSGGGGSAA |
| | | | | AMQSVLTOPPSASGTPGQRVTISCSGSSSNIGS |
| | | | | NYVYWYQQLPGTAPKLLIYRNNORPSGVPDRES |
| | | | | GSKSGTSASLAISGLRPEDEADYYCAAWDDSLC |
| | | | | VALVFGGGTNGGGGVDGTAG |
| 11 | 93 | JB1-QBP1 | 91 | MGKDYYQTLGLARGASDEEIKRAYRROALRYHP |
| | | | | DKNKEPGAEEKFKEIAEAYDVLSDPRKREIFDR |
| | | | | YGEEGLKGSGGGGSSNWKWWPGIFD |
| 12 | 94 | JB1(P33Q)- | 91 | MGKDYYQTLGLARGASDEEIKRAYRRQALRYHQ |
| | | QBP1 | | DKNKEPGAEEKFKEIAEAYDVLSDPRKREIFDR |
| | | | | YGEEGLKGSGGGGSSNWKWWPGIFD |
| 14 | 95 | JB1-TBP2 | 106 | MGKDYYQTLGLARGASDEEIKRAYRROALRYHP |
| | | | | DKNKEPGAEEKFKEIAEAYDVLSDPRKREIFDR |
| | | | | YGEEGLKGSDIGGGGSGGGGSGGGGSGGGGSAA |
| | | | | AVQIINK |
| 15 | 96 | JB1-TBP3 | 113 | MGKDYYQTLGLARGASDEEIKRAYRRQALRYHP |
| | | | | DKNKEPGAEEKFKEIAEAYDVLSDPRKREIFDR |
| | | | | YGEEGLKGSDIGGGGSGGGGSGGGGSGGGGSAA |
| | | | | AYQQYQDATADEQG |
| 16 | 97 | JB1-TBP1 | 86 | MGKDYYQTLGLARGASDEEIKRAYRRQALRYHP |
| | | (no linker) | | DKNKEPGAEEKFKEIAEAYDVLSDPRKREIFDR |
| | | | | YGEEGLKGSGGGGSVQIVYK |
| 17 | 98 | JB1-TBP2 | 86 | MGKDYYQTLGLARGASDEEIKRAYRRQALRYHP |
| | | (no linker) | | DKNKEPGAEEKFKEIAEAYDVLSDPRKREIFDR |
| | | | | YGEEGLKGSGGGGSVQIINK |
| 18 | 99 | JB1-TBP3 | 93 | MGKDYYQTLGLARGASDEEIKRAYRRQALRYHP |
| | | (no linker) | | DKNKEPGAEEKFKEIAEAYDVLSDPRKREIFDR |
| | | | | YGEEGLKGSGGGGSYQQYQDATADEQG |
| 19 | 100 | JB1- | 314 | MGKDYYQTLGLARGASDEEIKRAYRRQALRYHP |
| | | scFv(Tau) | | DKNKEPGAEEKFKEIAEAYDVLSDPRKREIFDR |
| | | (no linker) | | YGEEGLKGSGGGGSEVOLQQSGAELVOPGASVK |
| | | | | LSCTASGFNIKDTSMHWVRORPEQGLEWIGRIA |
| | | | | PANGNTKYDPKFQGKATITTDTSSNTAYLQLSS |
| | | | | LTSEDTAVYYCSGSGNYDWGQGTTLTVSGGGGS |
| | | | | GGGGSGGGGSDIQMNQSPSSLSASLGDTITISC |
| | | | | HASQNINVWLSWYQQKPGNIPKLLIYEASTLYT |
| | | | | GVPSRFSGSGSGTGFTLTISSLQPEDIATYYCQ |
| | | | | QGQSYPWTFGGGTKLEI |
| 20 | 101 | JB1-Happ1 | 198 | MGKDYYQTLGLARGASDEEIKRAYRRQALRYHP |
| | | (no linker) | | DKNKEPGAEEKFKEIAEAYDVLSDPRKREIFDR |
| | | | | YGEEGLKGSGGGGSMQSVLTQPPSASGTPGQRV |
| | | | | TISCSGSSSNIGSNYVYWYQQLPGTAPKLLIYR |
| | | | | NNQRPSGVPDRESGSKSGTSASLAISGLRPEDE |
| | | | | ADYYCAAWDDSLCVALVFGGGTNGGGGVDGTAG |

II. Nucleic Acids Encoding Fusion Protein Constructs

Another aspect of the invention provided are isolated nucleic acids comprising a

polynucleotide sequence selected from (a) a polynucleotide encoding the fusion protein of any of the foregoing embodiments, or (b) the complement of the polynucleotide of (a). The present invention provides isolated nucleic acids encoding fusion proteins comprising the J domain and tau-binding domain, and sequences complementary to such nucleic acid molecules encoding the fusion proteins, including homologous variants thereof. In another aspect, the invention encompasses methods to produce nucleic acids encoding the fusion proteins disclosed herein, and sequences complementary to the nucleic acid molecules encoding fusion proteins, including homologous variants thereof. The nucleic acid according to this aspect of the invention can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In yet another aspect, disclosed is a method of producing a fusion protein comprising (a) synthesizing and/or assembling nucleotides encoding the fusion protein, (b) incorporating the encoding gene into an expression vector appropriate for a host cell, (c) transforming the appropriate host cell with the expression vector, and (d) culturing the host cell under conditions causing or permitting the fusion protein to be expressed in the transformed host cell, thereby producing the biologically-active fusion protein, which is recovered as an isolated fusion protein by standard protein purification methods known in the art. Standard recombinant techniques in molecular biology is used to make the polynucleotides and expression vectors of the present invention.

In accordance with the invention, nucleic acid sequences that encode the fusion proteins disclosed herein (or its complement) are used to generate recombinant DNA molecules that direct the expression of the fusion proteins in appropriate host cells. Several cloning strategies are suitable for performing the present invention, many of which are used to generate a construct that comprises a gene coding for a fusion protein of the present invention, or its complement. In some embodiments, the cloning strategy is used to create a gene that encodes a fusion protein of the invention, or their complement.

In certain embodiments, a nucleic acid encoding one or more fusion proteins is an RNA molecule, and can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In various embodiments, the nucleic acid is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a 5′ cap or modified 5′ cap and/or a 3′poly(A) sequence. As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from Rnases. The capping moiety can be modified to modulate functionality of mRNA such as its stability or efficiency of translation. In a particular embodiment, the mRNA comprises a poly(A) tail sequence of between about 50 and about 5000 adenines. In one embodiment, the mRNA comprises a poly (A) sequence of between about 100 and about 1000 bases, between about 200 and about 500 bases, or between about 300 and about 400 bases. In one embodiment, the mRNA comprises a poly (A) sequence of about 65 bases, about 100 bases, about 200 bases, about 300 bases, about 400 bases, about 500 bases, about 600 bases, about 700 bases, about 800 bases, about 900 bases, or about 1000 or more bases. Poly(A) sequences can be modified chemically or enzymatically to modulate mRNA functionality such as localization, stability or efficiency of translation.

As used herein, the terms “polynucleotide variant” and “variant” and the like refer to polynucleotides displaying substantial sequence similarity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

III. Vectors Comprising Nucleic Acids Encoding Fusion Proteins

Also provided is a vector comprising nucleic acid according to the invention. Such a vector preferably comprises additional nucleic acid sequences such as elements necessary for transcription/translation of the nucleic acid sequence encoding a phosphatase (for example, a promoter and/or terminator sequences). Said vectors can also comprise nucleic acid sequences coding for selection markers (for example an antibiotic) to select or maintain host cells transformed with said vector. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to an affected cell (e.g. neuronal cells) In one embodiment, the vector contains an in vitro synthesized or synthetically prepared mRNA encoding a fusion protein comprising a J domain and a tau-binding domain. Illustrative examples of non-viral vectors include, but are not limited to mRNA, plasmids (e.g., DNA plasmids or RNA plasmids), transposons, cosmids, and bacterial artificial chromosomes. The term “nucleic acid cassette” or “expression cassette” as used herein refers to genetic sequences within the vector, which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Illustrative examples of vectors include, but are not limited to, a plasmid, autonomously replicating sequences, and transposable elements, e.g., piggyBac, Sleeping Beauty, Mosl, Tcl/mariner, Tol2, mini-Tol2, Tc3, MuA, Himar I, Frog Prince, and derivatives thereof. Additional Illustrative examples of vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Illustrative examples of viruses useful as vectors include, without limitation, retrovirus (including lentivirus), adenovirus, adeno-associated virus, herpesvirus (e.g., herpes simplex vims), poxvirus, baculovirus, papillomavirus, and papovavirus (e.g., SV40). Illustrative examples of expression vectors include, but are not limited to, pClneo vectors (Promega) for expression in mammalian cells; pLenti4/V 5-DEST™, pLenti6/V 5-DEST™, and pLenti6.2/V 5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

Illustrative ubiquitous expression control sequences suitable for use in particular embodiments include gene promoters, but are not limited to, a cytomegalovirus (CMV) immediate early promoter, a viral simian virus 40 (SV40) promoter (e.g. early or late), a Moloney murine leukemia virus (MoMLV) LTR promoter, a Rous sarcoma virus (RSV) LTR, a herpes simplex virus (HSV) (thymidine kinase) promoter, H5, P7.5, and PI I promoters from vaccinia virus, an elongation factor 1-alpha (Efla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), b-kinesin (b-KIN), the human ROSA 26 locus (Irions et al, Nature Biotechnology 25, 1477-1482 (2007)), a Ubiquitin C promoter (UBC), a phosphogly cerate kinase-1 (PGK) promoter, a cytomegalovirus enhancer/chicken b-actin (CAG) promoter, a b-actin promoter and a myeloproliferative sarcoma virus enhancer, negative control region deleted, dl587rev primer binding site substituted (MND) U3 promoter (Haas et al., Journal of Virology. 2003; 77(17): 9439-9450).

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” (SSR) refer to a particular nucleic acid sequence to which a recombinase recognizes and binds.

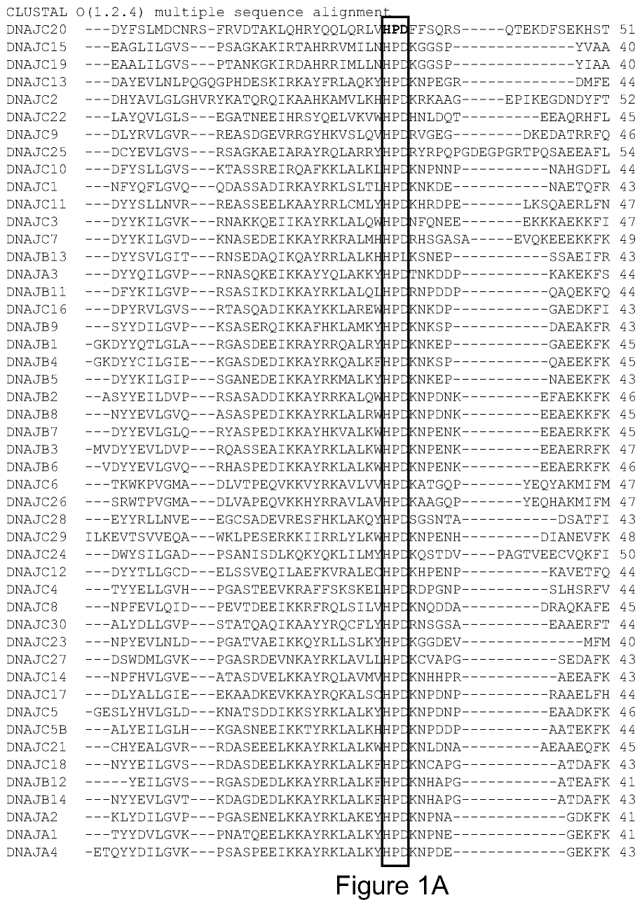

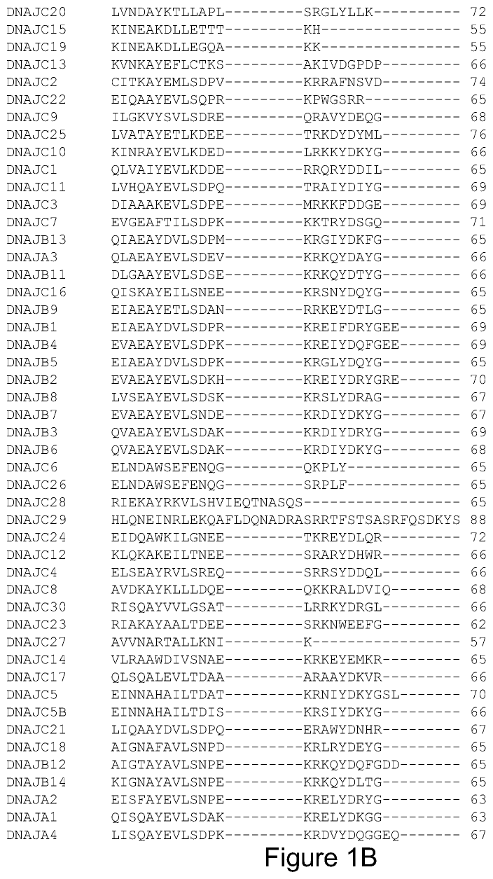

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Suitable recognition sites for the FLP recombinase include, but are not limited to: FRT (McLeod, et al., 1996), F1, F2, F3 (Schlake and Bode, 1994), FyFs (Schlake and Bode, 1994), FRT(LE) (Senecoff et al., 1988), and FRT(RE) (Senecoff et al., 1988).

Other examples of recognition sequences are the attB, attP, attL, and attR sequences, which are recognized by the recombinase enzyme I Integrase, e.g., phi-c31. The pC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by ϕ031 homodimers (Groth et al., 2000). The product sites, attL and attR, are effectively inert to further tpQA 1-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a fusion protein comprising a J domain and tau-binding domain. Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed.

IV. Delivery

In particular embodiments, one or more polynucleotides encoding a fusion protein comprising a J domain and tau-binding domain are introduced into a cell by non-viral or viral vectors. Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid nucleic acid conjugates, naked DNA, artificial virions, DEAE-dextran-mediated transfer, gene gun, and heat-shock.

Illustrative examples of polynucleotide delivery systems suitable for use in the particular embodiments contemplated include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al., (2003) Gene Therapy. 10: 180-187; and Balazs et al., (20W) Journal of Drug Delivery. 2011:1-12.

Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion), by intrathecal injection, intracerebroventricular injection or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding a fusion protein disclosed herein is administered directly to an organism for transduction of cells in vivo.

A viral vector, suitably packaged and formulated, can be delivered into the CNS via intrathecal delivery. For example, adeno-associated viral vectors can be delivered using methods described in U.S. Ser. No. 15/771,481, which is incorporated herein by reference in its entirety.

Alternatively, naked DNA can be administered. Administration is by any of the routes normally used for introducing a molecule into ultimate contact with blood or tissue cells including, but not limited to, injection, infusion, topical application, and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding fusion protein comprising a J domain and a tau-binding domain are introduced into a cell, e.g., a neuronal cell, by transducing the cell with a recombinant adeno-associated virus (rAAV), comprising the one or more polynucleotides. AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, AAVrh10 or AAV 10.

In some embodiments, a chimeric rAAV is used; the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, AAVrh10 or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety. In various embodiments, one or more polynucleotides encoding a fusion protein disclosed herein are introduced into a neuronal cell or neuronal stem cell, by transducing the cell with a retrovirus, e.g., lentivirus, comprising the one or more polynucleotides. As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex vims (HSV) (thymidine kinase) promoters. In certain embodiments, lentiviral vectors are produced according to known methods. See e.g., Kutner et al., BMC Biotechnol. 2009; 9:10. Doi: 10.1186/1472-6750-9-10; Kutner et al., Nat. Protoc. 2009; 4(4):495-505. Doi: 10.1038/nprot.2009.22.

According to certain specific embodiments contemplated herein, most or all of the viral vector backbone sequences are derived from a lentivirus, e.g., HIV-I. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a fusion protein disclosed herein are introduced into a target cell by transducing the cell with an adenovirus comprising the one or more polynucleotides. Adenoviral (Ad) based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding a fusion protein of the invention are introduced into the target cell of a subject by transducing the cell with a herpes simplex virus, e.g., HSV-I, HSV-2, encoding one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, or a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which is incorporated by reference herein in its entirety.

V. Cells Expressing the Fusion Protein

In yet another aspect, the invention provides for cells expressing the fusion proteins described herein. Cells can be transfected with a vector encoding the fusion protein as described herein above. In one embodiment, the cell is a prokaryotic cell. In another embodiment, the cell is a eukaryotic cell. In still another embodiment, the cell is a mammalian cell. In a particular embodiment, the cell is a human cell. In another embodiment, the cell is a human cell that is derived from a patient that suffers from, or is at risk of suffering from, a tau-mediated disorder including, but not limited to, ALS, FTD and Alzheimer's Disease. The cell can be a neuronal cell or a muscle cell.

Cells expressing the fusion protein can be useful in producing the fusion protein. In this embodiment, the cells are transfected with a vector for overexpressing the fusion protein. The fusion protein may optionally contain an epitope, for example, a human Fc domain or a FLAG epitope, as described herein above, that would facilitate the purification (using a Protein A- or anti-FLAG antibody column, respectively). The epitope may be connected to the rest of the fusion protein via a linker or a protease substrate sequence such that, during or after purification, the epitope can be removed from the fusion protein.

Cells expressing the fusion protein can also be useful in a therapeutic context. In one embodiment, cells are collected from a patient in need of therapy (e.g., a patient who suffers from or is at risk of suffering from a tau-mediated disorder). In one embodiment, the cells are neuronal cells. Collected cells are then transfected with a vector encoding the polynucleotide to express the fusion protein. The transfected cells can then be processed to enrich or select for transfected cells. The transfected cells can also be treated to differentiate into a different type of cell, for example, a neuronal cell. After processing, the transfected cells can be administered to the patient. In one embodiment, the cells are administered by directed injection into the CNS by intrathecal injection, intracranial injection or intracerebroventricular injection.

In an alternative embodiment, cells expressing a secreted form of the fusion protein can be used. For example, fusion protein constructs can be designed having a secretion signal sequence on the N-terminal end. Representative signal sequences are shown below in Table 7.

TABLE 7
Representative Signal Sequences

| SEQ ID NO: | SEQUENCE |
|---|---|
| 77 | MGVKVLFALICIAVAEA |
| 78 | MAPVQLLGLLVLFLPAMRC |
| 79 | MAVLGLLFCLVTFPSCVLS |

Therefore, in one embodiment, the fusion protein comprises a signal sequence and a fusion protein, wherein the signal sequence is selected from the group consisting of SEQ ID NOs: 77-79, and the fusion protein is selected from the group consisting of SEQ ID NOs: 83-88 and 95-101. In another embodiment, the fusion protein comprises the signal sequence of SEQ ID NO: 77, and the fusion protein selected from the group consisting of SEQ ID NOs: 83-88 and 95-101. In another embodiment, the fusion protein comprises the signal sequence of SEQ ID NO: 78, and the fusion protein selected from the group consisting of SEQ ID NOs: 83-88 and 95-101. In another embodiment, the fusion protein comprises the signal sequence of SEQ ID NO: 79, and the fusion protein selected from the group consisting of SEQ ID NOs: 83-88 and 95-101. Cells expressing the fusion protein constructs with the signal sequence can be administered to a subject, for example a human subject (e.g., a patient having or at risk of suffering from a tau disorder). The fusion protein is secreted from the cells, which help reduce tau protein aggregation and/or associated cytotoxicity.

As described above, in certain embodiments, the fusion protein can further comprise a cell-penetrating peptide. A cell expressing a fusion protein comprising a signal sequence and a cell-penetrating peptide would be capable of secreting the fusion protein, devoid of the signal sequence. The secreted fusion protein, also comprising the cell-penetrating peptide, would then be capable of entering nearby cells, and have the potential to reduce aggregation and/or cytotoxicity mediated by tau proteins in those cells. The fusion protein containing both a signal sequence and a cell-penetrating peptide and would be secreted via the signal sequence and be capable of entering cells via the cell-penetrating peptide sequence.

VI. Methods of Use

In another aspect, the invention provides a method for achieving a beneficial effect in tau disorders and/or in a tau disorder or condition mediated by tau aggregation. The tau disorder is selected from the group consisting of amyotrophic lateral sclerosis (ALS), frontotemporal dementia (FTD), Parkinson's disease, Huntington's disease, Alzheimer's disease, hippocampal sclerosis, and dementia with Lewy's bodies.

In some embodiments, the invention provides methods for treating a subject, such as a human, with a tau disease, disorder or condition comprising the step of administering to the subject a therapeutically- or prophylactically-effective amount of a fusion protein, a nucleic acid encoding such fusion protein, or a viral vector encoding such fusion protein described herein, wherein said administration results in the improvement of one or more biochemical or physiological parameters or clinical endpoints associated with the tau disease, disorder or condition.

In other embodiments, the invention provides for a method of reducing aggregation of tau in a cell. The cell can be a cultured cell or an isolated cell. The cell can also be derived from a subject, for example, a human subject. In one embodiment, the cell is in the CNS of the human subject. In another embodiment, the human subject is suffering from, or is at risk of suffering from a tau disorder disease, including, but not limited to, amyotrophic lateral sclerosis (ALS), frontotemporal dementia (FTD) and Alzheimer's Disease. In one particular embodiment, the tau disorder is Alzheimer's Disease.

Pathogenic tau proteins can be detected in a number of ways. In one example, hyperphosphorylated tau proteins can be distinguished from non-pathogenic (i.e., functional) tau, for example, by using an antibody that targets phosphorylated-tau or a conformation-specific tau. A greater reduction in the pathogenic tau protein, when compared with controls indicates a higher potency. Reduction of pathogenic tau proteins can also be detected directly in the cell, for example, using immunofluorescence microscopy with labeled reagents detecting the tau protein (see, for example, Ding et al., (2015) Oncotarget, 6: 24178-24191; Chou et al., (2015) Hum. Mol. Genet. 24:5154-5173, and Example 1). In certain embodiments, a greater reduction of tau polypeptide levels when compared with controls indicates a higher potency.

Therefore, in one embodiment, the method comprises contacting the cell with an effective amount of the fusion protein or a nucleic acid, vector, or viral particle encoding the fusion protein to reduce pathogenic tau proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

As shown below in Example 1, expression of fusion proteins comprising a J domain and a tau-binding domain has been found to reduce the overall level of tau reporter constructs. As such, in another embodiment, the method comprises contacting the cell with an amount of the fusion protein, a cell expressing the fusion protein, a nucleic acid, vector, or viral particle encoding the fusion protein effective to reduce the level of tau proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

VII. Pharmaceutical Compositions

The compositions contemplated herein may comprise one or more fusion protein comprising a J domain and tau-binding domain, polynucleotides encoding such fusion proteins, vectors comprising same, genetically modified cells, etc., as contemplated herein. Compositions include, but are not limited to pharmaceutical compositions. A “pharmaceutical composition” refers to a composition formulated in pharmaceutically acceptable or physiologically acceptable solutions for administration to a cell or subject, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, are suitable for use in contact with the tissues of human beings and animals without excessive toxicity, irritation, allergic response, or other problem or complication, commensurate with a reasonable benefit/risk ratio.

As used herein “pharmaceutically acceptable carrier”, “diluent” or “excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

VIII. Dosages

The dosage of the compositions (e.g., a composition including a fusion protein construct, nucleic acid or gene therapy viral particle) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the subject to be treated. The compositions described herein can be administered initially in a suitable dosage that can be adjusted as required, depending on the clinical response. In some aspects, the dosage of a composition is a prophylactically or therapeutically effective amount.

IX. Kits

Kits including (a) a pharmaceutical composition including a fusion protein construct, nucleic acid encoding such fusion protein, or viral particle encompassing such nucleic acid that reduces aggregation of tau proteins in a cell or subject described herein, and (b) a package insert with instructions to perform any of the methods described herein are contemplated. In some aspects, the kit includes (a) a pharmaceutical composition including a composition described herein that reduces the aggregation of tau proteins in a cell or subject described herein, (b) an additional therapeutic agent, and (c) a package insert with instructions to perform any of the methods described herein.

### Examples

To test whether J domains can be specifically engineered to facilitate the proper folding of aggregated proteins, we designed and tested a number of fusion protein constructs designed to target tau proteins.

### Example 1: Fusion Protein Design

A. Methods

### General Techniques and Materials

The practice of the present invention employs, unless otherwise indicated, conventional techniques of immunology, biochemistry, chemistry, molecular biology, microbiology, cell biology, genomics and recombinant DNA, which are known by persons with ordinary skill in the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al., eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4^{th} edition, John Wiley & Sons, Somerset, N J, 2000, the contents of which are incorporated in their entirety herein by reference. HEK-293 cells (human embryonic kidney cells) were purchased from the American Type Culture Collection (Manassas, VA). Anti-FLAG antibody was purchased from Thermo Fisher Scientific. For ease of purification, detection and/or characterization, some of the fusion protein constructs used in this Example 1 contain, in addition to the sequences provided in SEQ ID NOs: 83-101, the FLAG epitope of SEQ ID NO:67 at either the C-terminus or N-terminus of the protein, in addition to a short linker sequence.

### Expression and Detection of Proteins in HEK293 Cells

Expression vector plasmids encoding various protein constructs were transfected into HEK293 cells with Lipofectamine 3000 transfection reagent (Thermo Fisher Scientific). Cell lysates were analyzed for expressed proteins using immunoblot assays. Samples of culture media were centrifuged to remove debris prior to analysis. Cells were lysed in a lysis buffer (10 mM Tris-HCl, pH 8.0, 150 mM NaCl, 10 mM EDTA, 2% SDS) containing 2 mM PMSF and protease cocktail (Complete Protease Inhibitor Cocktail; Sigma). After brief sonication, the samples were analyzed for expressed proteins using immunoblot assays. For immunoblot analysis, samples were boiled in an SDS-sample buffer and run on polyacrylamide gel electrophoresis. Thereafter, the separated protein bands were transferred to a PVDF membrane.

Expressed proteins were detected using a chemiluminescent signal. Briefly, blots were incubated with a primary antibody capable of binding the particular epitope (e.g., anti-tau antibody). After rinsing away the unbound primary antibody, a secondary, enzyme-linked antibody (e.g., HRP-linked anti-IgG antibody) was allowed to bind with the primary antibody molecules bound to the blots. Following rinsing, a chemiluminescent reagent was added, and the resultant chemiluminescent signals in the blots were captured on X-ray film. The following antibodies were used:
anti-Tau antibody (MilliporeSigma, Burlington, MA),
anti-V5 antibody (Thermo Fisher Scientific),
anti-Flag antibody (Thermo Fisher Scientific),
anti-pTau(Ser396) antibody (BioLegend, San Diego, CA),
anti-pTau(Thr231) (BioLegend, San Diego, CA).

### Reporter Constructs

We first investigated whether the fusion proteins targeting tau ameliorates its aggregation in cultured cells. To this end, we generated two constructs expressing the wildtype tau (ON4R), as well as a mutant form (containing a P243S substitution) known to cause hyperphosphorylation and aggregation (See Table 8 below), fused on its N-terminus with the V5 epitope. HEK293 cells were cultured and transfected with the plasmids encoding the wildtype (SEQ ID NO: 80) or mutant (SEQ ID NO: 81) tau.

TABLE 8
Tau Reporter Constructs

| Construct | SEQ ID | | |
|---|---|---|---|
| Name | NO: | Length | Sequence |
| V5-Tau(0N4R) | 80 | 404 | MGKPIPNPLLGLDSTGTGSEFMAEPRQEFEVMEDHA |
| | | | GTYGLGDRKDQGGYTMHQDQEGDTDAGLKAEEAGIG |
| | | | DTPSLEDEAAGHVTQARMVSKSKDGTGSDDKKAKGA |
| | | | DGKTKIATPRGAAPPGQKGQANATRIPAKTPPAPKT |
| | | | PPSSGEPPKSGDRSGYSSPGSPGTPGSRSRTPSLPT |
| | | | PPTREPKKVAVVRTPPKSPSSAKSRLQTAPVPMPDL |
| | | | KNVKSKIGSTENLKHQPGGGKVQIINKKLDLSNVQS |
| | | | KCGSKDNIKHVPGGGSVQIVYKPVDLSKVTSKCGSL |
| | | | GNIHHKPGGGQVEVKSEKLDFKDRVQSKIGSLDNIT |
| | | | HVPGGGNKKIETHKLTFRENAKAKTDHGAEIVYKSP |
| | | | VVSGDTSPRHLSNVSSTGSIDMVDSPQLATLADEVS |
| | | | ASLAKQGL |
| V5-Tau(0N4R: | 81 | 404 | MGKPIPNPLLGLDSTGTGSEFMAEPRQEFEVMEDHA |
| P243S) | | | GTYGLGDRKDQGGYTMHQDQEGDTDAGLKAEEAGIG |
| | | | DTPSLEDEAAGHVTQARMVSKSKDGTGSDDKKAKGA |
| | | | DGKTKIATPRGAAPPGQKGQANATRIPAKTPPAPKT |
| | | | PPSSGEPPKSGDRSGYSSPGSPGTPGSRSRTPSLPT |
| | | | PPTREPKKVAVVRTPPKSPSSAKSRLQTAPVPMPDL |
| | | | KNVKSKIGSTENLKHQPGGGKVQIINKKLDLSNVQS |
| | | | KCGSKDNIKHVSGGGSVQIVYKPVDLSKVTSKCGSL |
| | | | GNIHHKPGGGQVEVKSEKLDFKDRVQSKIGSLDNIT |
| | | | HVPGGGNKKIETHKLTFRENAKAKTDHGAEIVYKSP |
| | | | VVSGDTSPRHLSNVSSTGSIDMVDSPQLATLADEVS |
| | | | ASLAKQGL |
| Tau3R | 82 | 241 | MIATPRGAAPPGQKGQANATRIPAKTPPAPKTPPSS |
| | | | GEPPKSGDRSGYSSPGSPGTPGSRSRTPSLPTPPTR |
| | | | EPKKVAVVRTPPKSPSSAKSRLQTAPVPMPDLKNVK |
| | | | SKIGSTENLKHQPGGGKVQIVYKPVDLSKVTSKCGS |
| | | | LGNIHHKPGGGQVEVKSEKLDFKDRVQSKIGSLDNI |
| | | | THVPGGGNKKIETHKLTFRENAKAKTDHGAEIVYKS |
| | | | PVVSGDTSPRHLSNVSSTGSIDMVD |

### Fusion Protein Constructs

Several fusion protein constructs were designed comprising the J domain from the human DnaJB1 (SEQ ID NO: 5), as provided in Table 6, and summarized below in Table 9. The transfected constructs in these experiments contained Flag epitope fusions on their C-terminal ends to facilitate detection. Controls for these fusion protein constructs include constructs containing the J domain with a P33Q mutation within the highly conserved HPD motif. In addition, further control constructs were designed, including a construct comprising only the human DnaJB1 J domain (SEQ ID NO: 5) without any tau-binding domain (Construct 13), (also with Flag epitope), a fusion protein construct (Construct 11) containing the DnaJB1 J domain fused on either side with the polyglutamine-binding peptide QBP1 as well as its corresponding control with the P33Q mutation within the J domain (Construct 12).

TABLE 9
Fusion Protein Constructs and Controls

| Construct | | SEQ ID | |
|---|---|---|---|
| No. | Construct Name | NO: | Notes |
| 1 | JB1-TBP | 83 | J domain from human DnaJB1 fused on its C- |
| | | | terminus to TBP (SEQ ID NO: 49) |
| 2 | TBP1-JB1-TBP1 | 84 | J domain from human DnaJB1 sandwiched |
| | | | between two TBP (SEQ ID NO: 49) peptides on |
| | | | either side. |
| 3 | JB1-2xTBP | 85 | J domain from human DnaJB1 fused on its C- |
| | | | terminus to tandem TBP (SEQ ID NO: 49) peptides |
| 4 | JB1-scFv(Tau) | 86 | J domain from human DnaJB1 fused on its C- |
| | | | terminus to scFv(Tau) (SEQ ID NO: 50) |
| 5 | JB1-scFv(MW7) | 87 | J domain from human DnaJB1 fused on its C- |
| | | | terminus to scFv(MW7) (SEQ ID NO: 51) |
| 6 | JB1-Happ1 | 88 | J domain from human DnaJB1 fused on its C- |
| | | | terminus to Happ1 (SEQ ID NO: 52) |
| 7 | JB1(P33Q)-TBP | 89 | J domain from human DnaJB1 containing a P33Q |
| | | | mutation fused on its C-terminus to TBP (SEQ ID |
| | | | NO: 49) |
| 8 | JB1(P33Q)- | 90 | J domain from human DnaJB1 containing a P33Q |
| | scFv(Tau) | | mutation fused on its C-terminus to scFv(Tau) |
| | | | (SEQ ID NO: 50) |
| 9 | JB1(P33Q)- | 91 | J domain from human DnaJB1 containing a P33Q |
| | scFv(MW7) | | mutation fused on its C-terminus to scFv(MW7) |
| | | | (SEQ ID NO: 51) |
| 10 | JB1(P33Q)-Happ1 | 92 | J domain from human DnaJB1 containing a P33Q |
| | | | mutation fused on its C-terminus to Happ1 |
| | | | (SEQ ID NO: 52) |
| 11 | QBP1-JB1-QBP1 | 93 | J domain from human DnaJB1 fused on its C- |
| | | | terminus to QBP1, a polyglutamine binding peptide |
| 12 | QBP1-JB1(P33Q)- | 94 | J domain from human DnaJB1 containing a P33Q |
| | QBP1 | | mutation fused on its C-terminus to QBP1, a |
| | | | polyglutamine binding peptide |
| 13 | JB1 only | 5 | A control construct comprising JB1 with no Tau |
| | | | binding domain |
| 14 | JB1-TBP2 | 95 | J domain from human DnaJB1 fused on its C-terminus |
| | | | to TBP2 (SEQ ID NO: 53) |
| 15 | JB1-TBP3 | 96 | J domain from human DnaJB1 fused on its C-terminus |
| | | | to TBP3 (SEQ ID NO: 54) |
| 16 | JB1-TBP (no | 97 | Similar to Construct 1, but devoid of linker |
| | linker) | | sequences between DnaJB1 and TBP |
| 17 | JB1-TBP2 (no | 98 | Similar to Construct 14, but devoid of linker |
| | linker) | | sequences between DnaJB1 and TBP2 |
| 18 | JB1-TBP3 (no | 99 | Similar to Construct 15, but devoid of linker |
| | linker) | | sequences between DnaJB1 and TBP |
| 19 | JB1-scFv(Tau) | 100 | Similar to Construct 4, but devoid of linker |
| | (no linker) | | sequences between DnaJB1 and TBP |
| 20 | JB1-Happ1 | 101 | Similar to Construct 6, but devoid of linker |
| | (no linker) | | sequences between DnaJB1 and TBP |

Several of these constructs were co-transfected along with constructs expressing wildtype or mutant tau into HEK293 cells. Expression of Construct 1, Construct 4, Construct 5 and Construct 6 were detected by immunoblot analysis (data not shown).

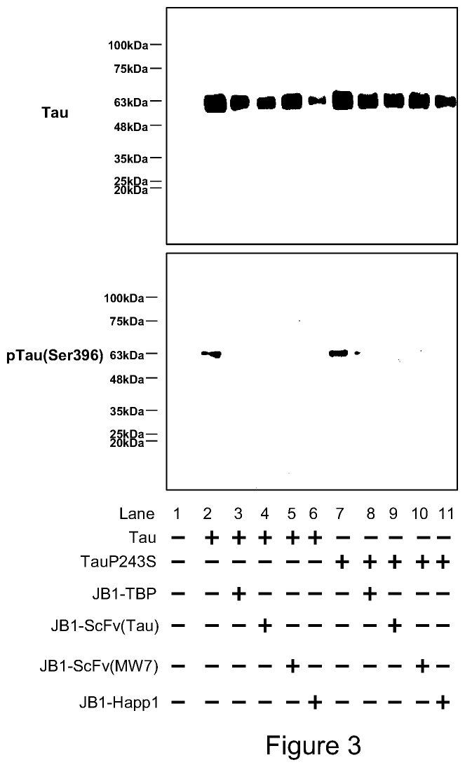

To determine whether J domains could be used to reduce tau aggregation, an initial experiment was conducted by co-expression of wildtype or mutant tau with a fusion protein comprising a J-domain sequence derived from a Hsp40 J-domain protein (from human DnaJB1), conjugated to the tau-binding peptide TBP (Construct 1), ScFv(Tau) (Construct 4), ScFv(MW7) (Construct 5) and Happ1 (Construct 6). As shown in FIG. 3, expression of either wildtype (FIG. 3, top panel, lanes 2-6) or mutant (P243S) tau (lanes 7-11) in HEK293 cells results in the appearance of tau as detected by anti-V5 antibodies. We further tested whether co-expression of one of the fusion protein constructs has an effect on altering the levels of phosphorylated tau. We therefore examined the amount of tau phosphorylated at Thr231 or Ser396 in Tau 2N4R, as hyperphosphorylation at these sites are associated with tauopathy (Bramblett et al., (1993) Neuron 10:1089-99; Alonso et al., (2004) J Biol Chem 279:34873-81; Lin et al., (2007) J Neurochem 103:802-13; Alonso et al., (2010) J Biol Chem 285:30851-30860). When probed with antibodies recognizing tau phosphorylated at Ser396 (2N4R) (FIG. 3, bottom panel), expression of wildtype or P243S tau alone (lanes 2 and 7, respectively) detects pTau(Ser396) at approximately 63 kDa. In contrast, the level of detectable pTau(Ser396) is dramatically reduced in extracts of cells co-expressing constructs 1, 4, 5, and 6 (lanes 3, 4, and 5 for cells expressing wildtype Tau, and lanes 9, 10, and 11 for cells expressing P243S Tau, respectively).

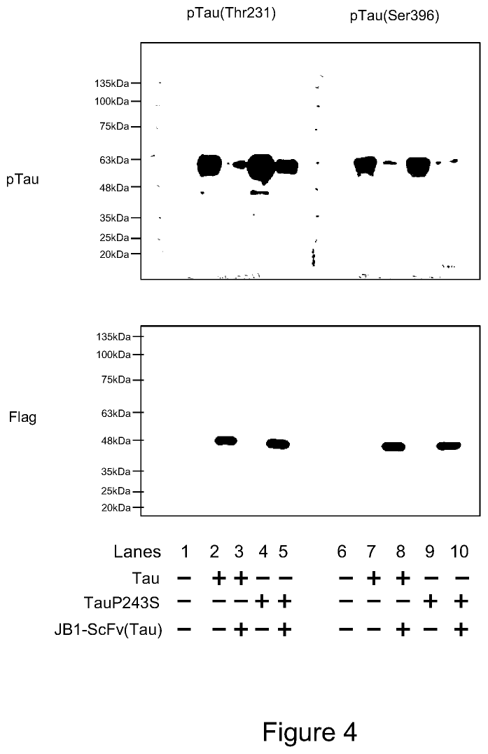

FIG. 4, top panel, shows that in cells expressing either wildtype (ON4R) or mutant Tau, co-expression of Construct 4 results in a dramatic reduction of pTau(Thr231 in 2N4R) and pTau(Ser396 in 2N4R). The bottom panel shows detection of Construct 4 using anti-Flag antibodies.

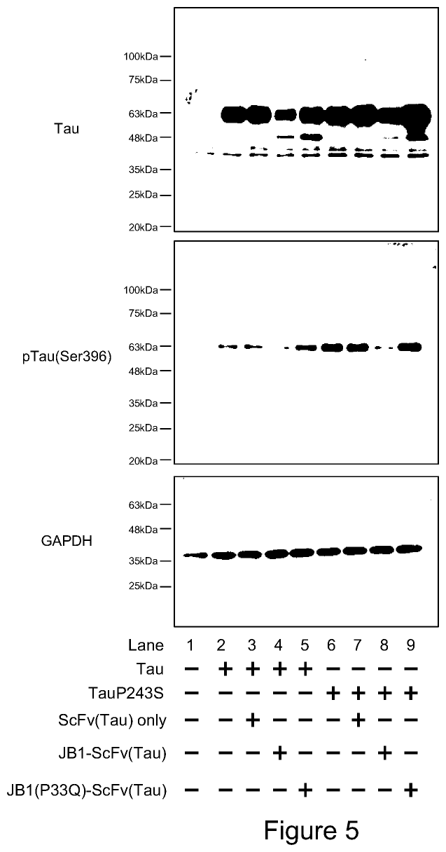

Finally, we tested the specificity of JB1-ScFv(Tau) in reducing pTau(Ser396). We therefore tested the ability of several constructs in their ability to reduce the amount of detectable pTau(Ser396), as shown in FIG. 5:
Construct 8, which is identical to Construct 4 with the exception of a P33Q mutation within the highly conserved HPD motif within the J domain (lanes 5 and 9);
ScFv(Tau) only control (Construct 4 without the J domain sequence; lanes 3 and 7)

As is shown in FIG. 5, expression of wildtype tau (lanes 2-5) or mutant (P243S) Tau (lanes 6-9) results in the appearance of detectable tau (top panel), as well as pTau(Ser396) (middle panel). The level of pTau(Ser396) is drastically reduced in cells co-expressing Construct 4 (lanes 4 and 8), but not in cells expressing Construct 8 (lanes 5 and 9) or ScFv(Tau) only (lanes 3 and 7). Therefore, we conclude that reduction in pTau(Ser396) is mediated by the fusion proteins such as Construct 4 requires functional J domain activity.

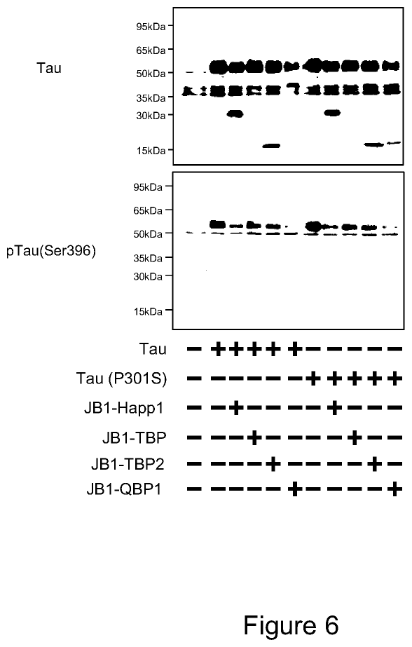

As is shown in FIG. 6, expression of wildtype Tau (lanes 2-6) or mutant (P243S) Tau (lanes 7-11) results in the appearance of detectable Tau (top panel), as well as pTau(Ser396) (bottom panel). The level of pTau(Ser396) is drastically reduced in cells co-expressing Construct 6 (lanes 3 and 8), Construct 1 (lanes 4 and 9), and Construct 14 (lanes 5 and 10), suggesting binding sequences such as TBP2 (SEQ ID NO: 53) is useful in the fusion construct.

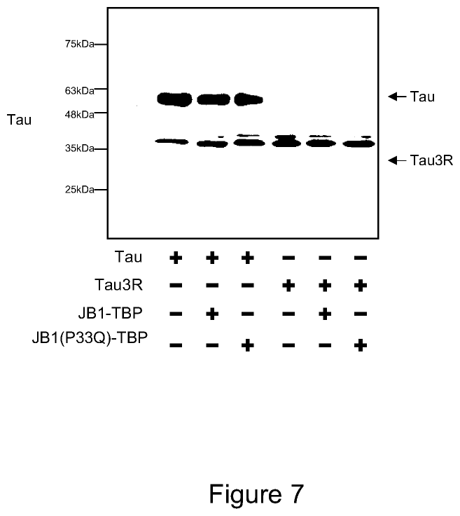

We further tested whether co-expression of one of the fusion protein constructs has an effect on altering the levels of the truncated form of tau (Tau3R, SEQ ID NO:80). The J-domain fusion protein with the tau-binding peptide TBP (Construct 1) was expressed with full length tau or truncated form of tau (Tau3R) in HEK293 cells. When probed with antibodies recognizing tau, full length tau was detected around 60 kDa while Tau3R was detected around 30 kDa (FIG. 7). Construct 1 significantly reduced the protein levels of Tau3R by mutating the J-domain (Construct 7) abolished the effects.

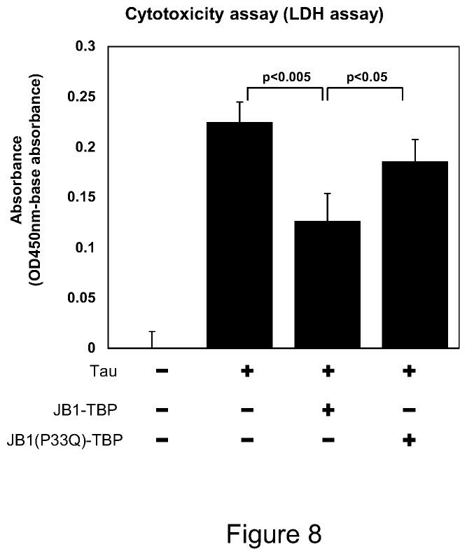

FIG. 8 shows the effect of expressing Construct 1 in U87-MG glioma cells. U87-MG glioma cells were infected with lentivirus to express Full length tau with or without Construct 1 or Construct 7. Culture medium was replaced with fresh medium at day 2. The culture medium was collected from U87-MG cells at 7-day after infection. Lactate dehydrogenase (LDH) activity in culture medium was measured by LDH-Cytox™ Assay Kit (BioLegend). Values represent the mean±SD. These results demonstrate that the J domain fusion proteins are capable of reducing not only protein misfolding and/or aggregation, but also cytotoxicity associated with protein misfolding and/or aggregation.

### Example 2: AAV Vectors Encoding Fusion Protein Constructs

An exemplary gene therapy vector is constructed by an AAVrh10 vector bearing codon-optimized cDNA encoding the fusion protein constructs of Table 6, specifically Constructs 1, 4, 5, and 6, as well as control Construct 13 (DnaJB1 J domain only), GFP (negative control), under the control of a CAG promoter, containing the cytomegalovirus (CMV) early enhancer element and the chicken beta-actin promoter. The cDNA encoding JB1-TBP is placed downstream of the Kozak sequence and upstream of the bovine growth hormone polyadenylation (BGHpA) signal. The entire cassette is flanked by two non-coding terminal inverted sequences of AAV-2.

Recombinant AAV vector is prepared using a baculovirus expression system similar to that described above (Urabe et al., 2002, Unzu et al., 2011 (reviewed in Kotin, 2011)). Briefly, three recombinant baculoviruses, one encoding REP for replication and packaging, one encoding CAP-5 for the capsid of AAVrh10, and one having an expression cassette is used to infect SF9 insect cells. Purification is performed using AVB Sepharose high speed affinity media (GE Healthcare Life Sciences, Piscataway, NJ). Vectors are titrated using QPCR with the primer-probe combination for the transgene and titers expressed as genomic copies per ml (GC/ml). The titer of the vector is approximately between 8×10¹³ to 2×10¹⁴ GC/ml.

### Example 3: Testing of Efficacy in a Mouse Model

Numerous animal models of Alzheimer's Disease exist (see, for example, Kitazawa et al. (2012) Curr. Pharm. Des., 18:1131-1147). In this example, the human tau (htau) mouse model of tauopathy is used (Duff et al., (2000) Neurobiol Dis. 7:87-98). Htau mice express human tau derived from a human PAC, H1 haplotype, known as 8c mice, while murine tau is knocked out by a targeted disruption of exon 1 (Duff et al., ibid). Mice are bred on a C57BL/6 background.

A preventive study is performed treating mice from 2.5 to 6.5 months of age. The different AAV viral particles containing vectors encoding the fusion proteins and corresponding controls are administered to the transgenic animal. In one embodiment, the viral particles are administered by tail vein injection. In another embodiment, the viral particles are administered by intramuscular injection. In still another embodiment, the particles are administered by intracranial injection, for example as described in Stanek et al., (2014) Hum. Gene. Ther. 25:461-474. Approximately 36 mice are divided into three groups of mixed male and female mice that are administered AAVrh10 harboring the cDNA encoding Construct 4, vector control, Construct 8 which has a P33Q mutation within the conserved HPD motif of the J domain.

After administration, disease progression is monitored and compared with control injected mice. The primary endpoint of the study is a statistically significant reduction of insoluble tau aggregates in the brains of the construct treated mice compared to the vector control treated mice. The secondary endpoints are dose-dependent reduction of insoluble tau aggregates, reduction of phosphorylated tau, and reduction of soluble tau.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. An isolated fusion protein comprising a J domain of a J protein and a tau-binding domain.

2. The fusion protein of claim 1, wherein the J domain of a J protein is of eukaryotic origin.

3. The fusion protein of any of claims 1-2, wherein the J domain of a J protein is of human origin.

4. The fusion protein of any of claims 1-3, wherein the J domain of a J protein is cytosolically localized.

5. The fusion protein of any of claims 1-4, wherein the J domain of a J protein is selected from the group consisting of SEQ ID Nos: 1-48.

6. The fusion protein of any of claims 1-5, wherein the J domain comprises the sequence selected from the group consisting of SEQ ID NOs: 5, 6, 10, 24, and 31.

7. The fusion protein of any of claims 1-6, wherein the J domain comprises the sequence of SEQ ID NO: 5.

8. The fusion protein of any of claims 1-6, wherein the J domain comprises the sequence of SEQ ID NO: 6.

9. The fusion protein of any of claims 1-6, wherein the J domain comprises the sequence of SEQ ID NO: 10.

10. The fusion protein of any of claims 1-6, wherein the J domain comprises the sequence of SEQ ID NO: 24.

11. The fusion protein of any of claims 1-6, wherein the J domain comprises the sequence of SEQ ID NO: 31.

12. The fusion protein of any of claims 1-11, wherein the tau-binding domain has a K_{D} for tau of 1 μM or less, for example, 300 nM or less, 100 nM or less, 30 nM or less, 10 nM or less when measured using an ELISA assay.

13. The fusion protein of any of claims 1-12, wherein the tau-binding domain comprises the sequence selected from the group consisting of SEQ ID NOs: 49-54.

14. The fusion protein of any of claims 1-13, wherein the tau-binding domain comprises the sequence of SEQ ID NO: 49.

15. The fusion protein of any of claims 1-13, wherein the tau-binding domain comprises the sequence of SEQ ID NO: 50.

16. The fusion protein of any of claims 1-13, wherein the tau-binding domain comprises the sequence of SEQ ID NO: 51.

17. The fusion protein of any of claims 1-16, comprising a plurality of tau-binding domains.

18. The fusion protein of any of claims 1-17, consisting of two tau-binding domains.

19. The fusion protein of any of claims 1-18, consisting of three tau-binding domains.

20. The fusion protein of any of claims 1-19, comprising one of the following constructs:

| | |
|---|---|
| a. | DNAJ-X-T, |
| b. | DNAJ-X-T-X-T, |
| c. | DNAJ-X-T-X-T-X-T, |
| d. | T-X-DNAJ, |
| e. | T-X-T-X-DNAJ, |
| f. | T-X-T-X-T-X-DNAJ, |
| g. | T-X-DNAJ-X-T, |
| h. | T-X-DNAJ-X-T-X-T, |
| i. | T-X-DNAJ-X-T-X-T-X-T, |
| j. | T-X-T-X-DNAJ-X-T, |
| k. | T-X-T-X-DNAJ-X-T-X-T, |
| l. | T-X-T-X-DNAJ-X-T-X-T-X-T, |
| m. | T-X-T-X-T-X-DNAJ-X-T, |
| n. | T-X-T-X-T-X-DNAJ-X-T-X-T, and |
| o. | T-X-T-X-T-X-DNAJ-X-T-X-T-X-T, |

wherein,
T is a tau-binding domain,
DNAJ is a J domain of a J protein, and
X is an optional linker.

21. The fusion protein of any of claims 1-20, wherein the fusion protein comprises the J domain sequence of SEQ ID NO: 5 and the tau-binding domain sequence of SEQ ID NO: 49.

22. The fusion protein of any of claims 1-21, wherein the fusion protein comprises the J domain sequence of SEQ ID NO: 5 and two copies of the tau-binding domain sequence of SEQ ID NO: 49.

23. The fusion protein of any of claims 1-22, wherein the fusion protein comprises the sequence selected from the group consisting of SEQ ID NOs: 83-88 and 95-101.

24. The fusion protein of any of claims 1-23, wherein the fusion protein comprises the sequence of SEQ ID NO: 83.

25. The fusion protein of any of claims 1-23, wherein the fusion protein comprises the sequence of SEQ ID NO: 87.

26. The fusion protein of any of claims 1-23, wherein the fusion protein comprises the sequence of SEQ ID NO: 88.

27. The fusion protein of any of claims 1-23, wherein the fusion protein comprises the sequence of SEQ ID NO: 97.

28. The fusion protein of any of claims 1-37, further comprising a targeting reagent.

29. The fusion protein of any of claims 1-28, further comprising an epitope.

30. The fusion protein of claim 29, wherein the epitope is a polypeptide selected from the group consisting of SEQ ID NOs: 66-72.

31. The fusion protein of any of claims 1-30, further comprising a cell-penetrating agent.

32. The fusion protein of claim 31, wherein the cell-penetrating agent comprises a peptide sequence selected from the group consisting of SEQ ID NOs: 73-76.

33. The fusion protein of any of claims 1-32, further comprising a signal sequence.

34. The fusion protein of claim 33, wherein the signal sequence comprises the peptide sequence selected from the group consisting of SEQ ID NOs: 77-79.

35. The fusion protein of any of claims 1-34, which is capable of reducing aggregation of tau proteins in a cell.

36. The fusion protein of any of claims 1-35, which is capable of reducing tau-mediated cytotoxicity.

37. A nucleic acid sequence encoding the fusion protein of any of claims 1-36.

38. The nucleic acid sequence of claim 37, wherein said nucleic acid is DNA.

39. The nucleic acid sequence of claim 37, wherein said nucleic acid is RNA.

40. The nucleic acid sequence of any of claims 37-39, wherein said nucleic acid comprises at least one modified nucleic acid.

41. A vector comprising the nucleic acid sequence of any of claims 37-40.

42. The vector of claim 41, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.

43. A virus particle comprising a capsid and the vector of claim 41 or claim 42.

44. The virus particle of claim 43, wherein the capsid is selected from the group consisting of AAV1, AAV2, AAV3, AAV4, AAV5, AAV6, AAV7, AAV8, AAV9, AAVrh10, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

45. A pharmaceutical composition comprising an agent selected from the group consisting of the fusion protein of any of claims 1-36, a cell expressing the fusion protein of claim 1-36, the nucleic acid of any of claims 37-40, the vector of any of claims 41-42, the virus particle of any of claims 43-44, and a pharmaceutically acceptable carrier or excipient.

46. A method of reducing toxicity of a tau protein in a cell, comprising contacting said cell with the fusion protein of any of claims 1-36, a cell expressing the fusion protein of claim 1-36, the nucleic acid of any of claims 37-40, the vector of any of claims 41-42, the virus particle of any of claims 43-44, and the pharmaceutically composition of claim 45.

47. The method of claim 46, wherein the cell is in a subject.

48. The method of any of claim 47, wherein the subject is a human.

49. The method of any one of claims 46-48, wherein the cell is located in the central nervous system.

50. The method of any one of claims 46-49, wherein the subject is identified as having a tau disease.

51. The method of claim 50, wherein the tau disease is selected from the group consisting of Alzheimer's Disease (AD), Parkinson's Disease (PD), Primary age-related tauopathy (PART), Chronic traumatic encephalopathy (CTE), Progressive supranuclear palsy (PSP), Corticobasal degeneration (CBD), Frontotemporal dementia and parkinsonism linked to chromosome 17 (FTDP-17), Lytico-bodig disease, Ganglioglioma and gangliocytoma, Meningioangiomatosis, Postencephalitic parkinsonism, Subacute sclerosing panencephalitis (SSPE), lead encephalopathy, tuberous sclerosis, Pantothenate kinase-associated neurodegeneration, and lipofuscinosis.

52. The method of claim 50 or claim 51, wherein the tau disease is Alzheimer's Disease.

53. The method of any one of claims 46-52, wherein there is a reduction in the amount of aggregated tau protein in the cell when compared with a control cell.

54. A method of treating, preventing, or delaying the progression of a tau disease in a subject in need thereof, the method comprising administering an effective amount of one or more agents selected from the group consisting of with the fusion protein of any of claims 1-36, a cell expressing the fusion protein of claims 1-36, the nucleic acid of any of claims 37-40, the vector of any of claims 41-42, the virus particle of any of claims 43-44, and the pharmaceutically composition of claim 45.

55. The method of claim 54, wherein the tau disease is selected from the group consisting of Alzheimer's Disease (AD), Parkinson's Disease (PD), Primary age-related tauopathy (PART), Chronic traumatic encephalopathy (CTE), Progressive supranuclear palsy (PSP), Corticobasal degeneration (CBD), Frontotemporal dementia and parkinsonism linked to chromosome 17 (FTDP-17), Lytico-bodig disease, Ganglioglioma and gangliocytoma, Meningioangiomatosis, Postencephalitic parkinsonism, Subacute sclerosing panencephalitis (SSPE), lead encephalopathy, tuberous sclerosis, Pantothenate kinase-associated neurodegeneration, and lipofuscinosis.

56. The method of claim 55, wherein the tau disease is Alzheimer's Disease.

57. Use of one or more of the fusion protein of any of claims 1-36, a cell expressing the fusion protein of any of claims 1-36, the nucleic acid of any of claims 37-40, the vector of any of claims 41-42, the virus particle of any of claims 43-44, and the pharmaceutically composition of claim 45, in preventing or delaying the progression of a tau disease in a subject.

58. Use of one or more of the fusion protein of any of claims 1-36, a cell expressing the fusion protein of any of claims 1-36, the nucleic acid of any of claims 37-40, the vector of any of claims 41-42, the virus particle of any of claims 43-44, and the pharmaceutically composition of claim 45, in the preparation of a medicament for the treatment or prevention of a Parkinson's disease in a subject.


SEQUENCE LISTING (101 sequences)

SEQ ID NO: 1 - protein, 63 aa, Homo Sapien
  ref <308>: DATABASE ACCESSION NUMBER: NCBI/NP_001530
  ref <309>: DATABASE ENTRY DATE: 2021-08-16
  ref <313>: RELEVANT RESIDUES IN SEQ ID NO: (6)..(68)
TYYDVLGVKPNATQEELKKAYRKLALKYHPDKNPNEGEKFKQISQAYEVLSDAKKRELYDKGG

SEQ ID NO: 2 - protein, 63 aa, Homo Sapien
  ref <308>: DATABASE ACCESSION NUMBER: NCBI/NP_005871
  ref <309>: DATABASE ENTRY DATE: 2021-06-26
  ref <313>: RELEVANT RESIDUES IN SEQ ID NO: (8)..(70)
KLYDILGVPPGASENELKKAYRKLAKEYHPDKNPNAGDKFKEISFAYEVLSNPEKRELYDRYG

SEQ ID NO: 3 - protein, 66 aa, Homo Sapien
  ref <308>: DATABASE ACCESSION NUMBER: NCBI/NP_001128582
  ref <309>: DATABASE ENTRY DATE: 2021-06-27
  ref <313>: RELEVANT RESIDUES IN SEQ ID NO: (93)..(158)
DYYQILGVPRNASQKEIKKAYYQLAKKYHPDTNKDDPKAKEKFSQLAEAYEVLSDEVKRKQYDAYG

SEQ ID NO: 4 - protein, 67 aa, Homo Sapien
  ref <308>: DATABASE ACCESSION NUMBER: NCBI/NP_001123654
  ref <309>: DATABASE ENTRY DATE: 2021-07-11
  ref <313>: RELEVANT RESIDUES IN SEQ ID NO: (4)..(70)
ETQYYDILGVKPSASPEEIKKAYRKLALKYHPDKNPDEGEKFKLISQAYEVLSDPKKRDVYDQGGEQ

SEQ ID NO: 5 - protein, 69 aa, Homo Sapien
  ref <308>: DATABASE ACCESSION NUMBER: NCBI/6Z5N_A
  ref <309>: DATABASE ENTRY DATE: 2020-12-29
  ref <313>: RELEVANT RESIDUES IN SEQ ID NO: (2)..(70)
GKDYYQTLGLARGASDEEIKRAYRRQALRYHPDKNKEPGAEEKFKEIAEAYDVLSDPRKREIFDRYGEE

SEQ ID NO: 6 - protein, 70 aa, Homo Sapien
  ref <308>: DATABASE ACCESSION NUMBER: NCBI/NP_001034639
  ref <309>: DATABASE ENTRY DATE: 2021-06-28
  ref <313>: RELEVANT RESIDUES IN SEQ ID NO: (2)..(71)
ASYYEILDVPRSASADDIKKAYRRKALQWHPDKNPDNKEFAEKKFKEVAEAYEVLSDKHKREIYDRYGRE

SEQ ID NO: 7 - protein, 69 aa, Homo Sapien
  ref <308>: DATABASE ACCESSION NUMBER: Uniprot/Q8WWF6
  ref <309>: DATABASE ENTRY DATE: 2021-09-04
  ref <313>: RELEVANT RESIDUES IN SEQ ID NO: (1)..(69)
MVDYYEVLDVPRQASSEAIKKAYRKLALKWHPDKNPENKEEAERRFKQVAEAYEVLSDAKKRDIYDRYG

SEQ ID NO: 8 - protein, 69 aa, Homo Sapien
  ref <308>: DATABASE ACCESSION NUMBER: NCBI/NP_001304028
  ref <309>: DATABASE ENTRY DATE: 2021-07-02
  ref <313>: RELEVANT RESIDUES IN SEQ ID NO: (2)..(70)
GKDYYCILGIEKGASDEDIKKAYRKQALKFHPDKNKSPQAEEKFKEVAEAYEVLSDPKKREIYDQFGEE

SEQ ID NO: 9 - protein, 65 aa, Homo Sapien
  ref <308>: DATABASE ACCESSION NUMBER: NCBI/NP_001128476
  ref <309>: DATABASE ENTRY DATE: 2021-07-11
  ref <313>: RELEVANT RESIDUES IN SEQ ID NO: (38)..(102)
DYYKILGIPSGANEDEIKKAYRKMALKYHPDKNKEPNAEEKFKEIAEAYDVLSDPKKRGLYDQYG

SEQ ID NO: 10 - protein, 68 aa, Homo Sapien
  ref <308>: DATABASE ACCESSION NUMBER: NCBI/NP_005485
  ref <309>: DATABASE ENTRY DATE: 2021-06-28
  ref <313>: RELEVANT RESIDUES IN SEQ ID NO: (2)..(69)
VDYYEVLGVQRHASPEDIKKAYRKLALKWHPDKNPENKEEAERKFKQVAEAYEVLSDAKKRDIYDKYG

SEQ ID NO: 11 - protein, 67 aa, Homo Sapien
  ref <308>: DATABASE ACCESSION NUMBER: NCBI/NP_660157
  ref <309>: DATABASE ENTRY DATE: 2021-06-27
  ref <313>: RELEVANT RESIDUES IN SEQ ID NO: (3)..(69)
DYYEVLGLQRYASPEDIKKAYHKVALKWHPDKNPENKEEAERKFKEVAEAYEVLSNDEKRDIYDKYG

SEQ ID NO: 12 - protein, 67 aa, Homo Sapien
  ref <308>: DATABASE ACCESSION NUMBER: NCBI/NP_699161
  ref <309>: DATABASE ENTRY DATE: 2021-07-12
  ref <313>: RELEVANT RESIDUES IN SEQ ID NO: (3)..(69)
NYYEVLGVQASASPEDIKKAYRKLALRWHPDKNPDNKEEAEKKFKLVSEAYEVLSDSKKRSLYDRAG

SEQ ID NO: 13 - protein, 65 aa, Homo Sapien
  ref <308>: DATABASE ACCESSION NUMBER: NCBI/NP_036460
  ref <309>: DATABASE ENTRY DATE: 2021-10-17
  ref <313>: RELEVANT RESIDUES IN SEQ ID NO: (26)..(90)
SYYDILGVPKSASERQIKKAFHKLAMKYHPDKNKSPDAEAKFREIAEAYETLSDANRRKEYDTLG

SEQ ID NO: 14 - protein, 66 aa, Homo Sapien
  ref <308>: DATABASE ACCESSION NUMBER: NCBI/NP_057390
  ref <309>: DATABASE ENTRY DATE: 2021-08-09
  ref <313>: RELEVANT RESIDUES IN SEQ ID NO: (26)..(91)
DFYKILGVPRSASIKDIKKAYRKLALQLHPDRNPDDPQAQEKFQDLGAAYEVLSDSEKRKQYDTYG

SEQ ID NO: 15 - protein, 65 aa, Homo Sapien
  ref <308>: DATABASE ACCESSION NUMBER: NCBI/NP_001002762
  ref <309>: DATABASE ENTRY DATE: 2021-07-02
  ref <313>: RELEVANT RESIDUES IN SEQ ID NO: (112)..(176)
YEILGVSRGASDEDLKKAYRRLALKFHPDKNHAPGATEAFKAIGTAYAVLSNPEKRKQYDQFGDD

SEQ ID NO: 16 - protein, 65 aa, Homo Sapien
  ref <308>: DATABASE ACCESSION NUMBER: NCBI/NP_705842
  ref <309>: DATABASE ENTRY DATE: 2021-01-25
  ref <313>: RELEVANT RESIDUES IN SEQ ID NO: (4)..(68)
DYYSVLGITRNSEDAQIKQAYRRLALKHHPLKSNEPSSAEIFRQIAEAYDVLSDPMKRGIYDKFG

SEQ ID NO: 17 - protein, 65 aa, Homo Sapien
  ref <308>: DATABASE ACCESSION NUMBER: NCBI/NP_001026893
  ref <309>: DATABASE ENTRY DATE: 2021-06-26
  ref <313>: RELEVANT RESIDUES IN SEQ ID NO: (108)..(172)
NYYEVLGVTKDAGDEDLKKAYRKLALKFHPDKNHAPGATDAFKKIGNAYAVLSNPEKRKQYDLTG

SEQ ID NO: 18 - protein, 65 aa, Homo Sapien
  ref <308>: DATABASE ACCESSION NUMBER: NCBI/NP_071760
  ref <309>: DATABASE ENTRY DATE: 2020-11-19
  ref <313>: RELEVANT RESIDUES IN SEQ ID NO: (65)..(129)
NFYQFLGVQQDASSADIRKAYRKLSLTLHPDKNKDENAETQFRQLVAIYEVLKDDERRQRYDDIL

SEQ ID NO: 19 - protein, 74 aa, Homo Sapien
  ref <308>: DATABASE ACCESSION NUMBER: NCBI/NP_001123359
  ref <309>: DATABASE ENTRY DATE: 2021-02-21
  ref <313>: RELEVANT RESIDUES IN SEQ ID NO: (88)..(161)
DHYAVLGLGHVRYKATQRQIKAAHKAMVLKHHPDKRKAAGEPIKEGDNDYFTCITKAYEMLSDPVKRRAFNSVD

SEQ ID NO: 20 - protein, 69 aa, Homo Sapien
  ref <308>: DATABASE ACCESSION NUMBER: NCBI/NP_006251
  ref <309>: DATABASE ENTRY DATE: 2021-06-26
  ref <313>: RELEVANT RESIDUES IN SEQ ID NO: (394)..(462)
DYYKILGVKRNAKKQEIIKAYRKLALQWHPDNFQNEEEKKKAEKKFIDIAAAKEVLSDPEMRKKFDDGE

SEQ ID NO: 21 - protein, 66 aa, Homo Sapien
  ref <308>: DATABASE ACCESSION NUMBER: NCBI/NP_001294909
  ref <309>: DATABASE ENTRY DATE: 2021-06-23
  ref <313>: RELEVANT RESIDUES IN SEQ ID NO: (33)..(98)
TYYELLGVHPGASTEEVKRAFFSKSKELHPDRDPGNPSLHSRFVELSEAYRVLSREQSRRSYDDQL

SEQ ID NO: 22 - protein, 70 aa, Homo Sapien
  ref <308>: DATABASE ACCESSION NUMBER: NCBI/NP_079495
  ref <309>: DATABASE ENTRY DATE: 2021-10-03
  ref <313>: RELEVANT RESIDUES IN SEQ ID NO: (13)..(82)
GESLYHVLGLDKNATSDDIKKSYRKLALKYHPDKNPDNPEAADKFKEINNAHAILTDATKRNIYDKYGSL

SEQ ID NO: 23 - protein, 66 aa, Homo Sapien
  ref <308>: DATABASE ACCESSION NUMBER: NCBI/NP_149096
  ref <309>: DATABASE ENTRY DATE: 2021-02-17
  ref <313>: RELEVANT RESIDUES IN SEQ ID NO: (19)..(84)
ALYEILGLHKGASNEEIKKTYRKLALKHHPDKNPDDPAATEKFKEINNAHAILTDISKRSIYDKYG

SEQ ID NO: 24 - protein, 65 aa, Homo Sapien
  ref <308>: DATABASE ACCESSION NUMBER: NCBI/NP_001243793
  ref <309>: DATABASE ENTRY DATE: 2021-05-09
  ref <313>: RELEVANT RESIDUES IN SEQ ID NO: (906)..(970)
TKWKPVGMADLVTPEQVKKVYRKAVLVVHPDKATGQPYEQYAKMIFMELNDAWSEFENQGQKPLY

SEQ ID NO: 25 - protein, 71 aa, Homo Sapien
  ref <308>: DATABASE ACCESSION NUMBER: NCBI/NP_001138238
  ref <309>: DATABASE ENTRY DATE: 2021-02-22
  ref <313>: RELEVANT RESIDUES IN SEQ ID NO: (325)..(395)
DYYKILGVDKNASEDEIKKAYRKRALMHHPDRHSGASAEVQKEEEKKFKEVGEAFTILSDPKKKTRYDSGQ

SEQ ID NO: 26 - protein, 68 aa, Homo Sapien
  ref <308>: DATABASE ACCESSION NUMBER: NCBI/NP_055095
  ref <309>: DATABASE ENTRY DATE: 2021-06-26
  ref <313>: RELEVANT RESIDUES IN SEQ ID NO: (57)..(124)
NPFEVLQIDPEVTDEEIKKRFRQLSILVHPDKNQDDADRAQKAFEAVDKAYKLLLDQEQKKRALDVIQ

SEQ ID NO: 27 - protein, 68 aa, Homo Sapien
  ref <308>: DATABASE ACCESSION NUMBER: NCBI/NP_056005
  ref <309>: DATABASE ENTRY DATE: 2021-07-25
  ref <313>: RELEVANT RESIDUES IN SEQ ID NO: (15)..(82)
DLYRVLGVRREASDGEVRRGYHKVSLQVHPDRVGEGDKEDATRRFQILGKVYSVLSDREQRAVYDEQG

SEQ ID NO: 28 - protein, 66 aa, Homo Sapien
  ref <308>: DATABASE ACCESSION NUMBER: NCBI/NP_001258510
  ref <309>: DATABASE ENTRY DATE: 2021-08-09
  ref <313>: RELEVANT RESIDUES IN SEQ ID NO: (35)..(100)
DFYSLLGVSKTASSREIRQAFKKLALKLHPDKNPNNPNAHGDFLKINRAYEVLKDEDLRKKYDKYG

SEQ ID NO: 29 - protein, 69 aa, Homo Sapien
  ref <308>: DATABASE ACCESSION NUMBER: NCBI/NP_060668
  ref <309>: DATABASE ENTRY DATE: 2021-04-17
  ref <313>: RELEVANT RESIDUES IN SEQ ID NO: (14)..(82)
DYYSLLNVRREASSEELKAAYRRLCMLYHPDKHRDPELKSQAERLFNLVHQAYEVLSDPQTRAIYDIYG

SEQ ID NO: 30 - protein, 66 aa, Homo Sapien
  ref <308>: DATABASE ACCESSION NUMBER: NCBI/NP_068572
  ref <309>: DATABASE ENTRY DATE: 2021-06-26
  ref <313>: RELEVANT RESIDUES IN SEQ ID NO: (14)..(79)
DYYTLLGCDELSSVEQILAEFKVRALECHPDKHPENPKAVETFQKLQKAKEILTNEESRARYDHWR

SEQ ID NO: 31 - protein, 66 aa, Homo Sapien
  ref <308>: DATABASE ACCESSION NUMBER: NCBI/NP_056083
  ref <309>: DATABASE ENTRY DATE: 2021-04-16
  ref <313>: RELEVANT RESIDUES IN SEQ ID NO: (1301)..(1366)
DAYEVLNLPQGQGPHDESKIRKAYFRLAQKYHPDKNPEGRDMFEKVNKAYEFLCTKSAKIVDGPDP

SEQ ID NO: 32 - protein, 65 aa, Homo Sapien
  ref <308>: DATABASE ACCESSION NUMBER: NCBI/NP_115740
  ref <309>: DATABASE ENTRY DATE: 2021-07-12
  ref <313>: RELEVANT RESIDUES IN SEQ ID NO: (443)..(507)
NPFHVLGVEATASDVELKKAYRQLAVMVHPDKNHHPRAEEAFKVLRAAWDIVSNAEKRKEYEMKR

SEQ ID NO: 33 - protein, 55 aa, Homo Sapien
  ref <308>: DATABASE ACCESSION NUMBER: NCBI/NP_037370
  ref <309>: DATABASE ENTRY DATE: 2021-04-17
  ref <313>: RELEVANT RESIDUES IN SEQ ID NO: (96)..(150)
EAGLILGVSPSAGKAKIRTAHRRVMILNHPDKGGSPYVAAKINEAKDLLETTTKH

SEQ ID NO: 34 - protein, 65 aa, Homo Sapien
  ref <308>: DATABASE ACCESSION NUMBER: NCBI/EAW51732
  ref <309>: DATABASE ENTRY DATE: 2015-03-23
  ref <313>: RELEVANT RESIDUES IN SEQ ID NO: (29)..(93)
DPYRVLGVSRTASQADIKKAYKKLAREWHPDKNKDPGAEDKFIQISKAYEILSNEEKRSNYDQYG

SEQ ID NO: 35 - protein, 66 aa, Homo Sapien
  ref <308>: DATABASE ACCESSION NUMBER: NCBI/NP_060633
  ref <309>: DATABASE ENTRY DATE: 2021-06-24
  ref <313>: RELEVANT RESIDUES IN SEQ ID NO: (11)..(76)
DLYALLGIEEKAADKEVKKAYRQKALSCHPDKNPDNPRAAELFHQLSQALEVLTDAAARAAYDKVR

SEQ ID NO: 36 - protein, 65 aa, Homo Sapien
  ref <308>: DATABASE ACCESSION NUMBER: NCBI/NP_689899
  ref <309>: DATABASE ENTRY DATE: 2020-12-11
  ref <313>: RELEVANT RESIDUES IN SEQ ID NO: (82)..(146)
NYYEILGVSRDASDEELKKAYRKLALKFHPDKNCAPGATDAFKAIGNAFAVLSNPDKRLRYDEYG

SEQ ID NO: 37 - protein, 55 aa, Homo Sapien
  ref <308>: DATABASE ACCESSION NUMBER: NCBI/NP_001177162
  ref <309>: DATABASE ENTRY DATE: 2021-02-23
  ref <313>: RELEVANT RESIDUES IN SEQ ID NO: (37)..(91)
EAALILGVSPTANKGKIRDAHRRIMLLNHPDKGGSPYIAAKINEAKDLLEGQAKK

SEQ ID NO: 38 - protein, 72 aa, Homo Sapien
  ref <308>: DATABASE ACCESSION NUMBER: NCBI/NP_741999
  ref <309>: DATABASE ENTRY DATE: 2021-09-20
  ref <313>: RELEVANT RESIDUES IN SEQ ID NO: (72)..(143)
DYFSLMDCNRSFRVDTAKLQHRYQQLQRLVHPDFFSQRSQTEKDFSEKHSTLVNDAYKTLLAPLSRGLYLLK

SEQ ID NO: 39 - protein, 67 aa, Homo Sapien
  ref <308>: DATABASE ACCESSION NUMBER: NCBI/NP_001012339
  ref <309>: DATABASE ENTRY DATE: 2021-06-25
  ref <313>: RELEVANT RESIDUES IN SEQ ID NO: (3)..(69)
CHYEALGVRRDASEEELKKAYRKLALKWHPDKNLDNAAEAAEQFKLIQAAYDVLSDPQERAWYDNHR

SEQ ID NO: 40 - protein, 65 aa, Homo Sapien
  ref <308>: DATABASE ACCESSION NUMBER: NCBI/NP_001291873
  ref <309>: DATABASE ENTRY DATE: 2021-02-16
  ref <313>: RELEVANT RESIDUES IN SEQ ID NO: (277)..(341)
LAYQVLGLSEGATNEEIHRSYQELVKVWHPDHNLDQTEEAQRHFLEIQAAYEVLSQPRKPWGSRR

SEQ ID NO: 41 - protein, 62 aa, Homo Sapien
  ref <308>: DATABASE ACCESSION NUMBER: NCBI/NP_009145
  ref <309>: DATABASE ENTRY DATE: 2021-10-17
  ref <313>: RELEVANT RESIDUES IN SEQ ID NO: (104)..(165)
NPYEVLNLDPGATVAEIKKQYRLLSLKYHPDKGGDEVMFMRIAKAYAALTDEESRKNWEEFG

SEQ ID NO: 42 - protein, 72 aa, Homo Sapien
  ref <308>: DATABASE ACCESSION NUMBER: NCBI/NP_859057
  ref <309>: DATABASE ENTRY DATE: 2021-02-16
  ref <313>: RELEVANT RESIDUES IN SEQ ID NO: (11)..(82)
DWYSILGADPSANISDLKQKYQKLILMYHPDKQSTDVPAGTVEECVQKFIEIDQAWKILGNEETKREYDLQR

SEQ ID NO: 43 - protein, 76 aa, Homo Sapien
  ref <308>: DATABASE ACCESSION NUMBER: NCBI/NP_001015882
  ref <309>: DATABASE ENTRY DATE: 2020-12-12
  ref <313>: RELEVANT RESIDUES IN SEQ ID NO: (55)..(130)
DCYEVLGVSRSAGKAEIARAYRQLARRYHPDRYRPQPGDEGPGRTPQSAEEAFLLVATAYETLKDEETRKDYDYML

SEQ ID NO: 44 - protein, 65 aa, Homo Sapien
  ref <308>: DATABASE ACCESSION NUMBER: NCBI/NP_001305063
  ref <309>: DATABASE ENTRY DATE: 2021-06-29
  ref <313>: RELEVANT RESIDUES IN SEQ ID NO: (1168)..(1232)
SRWTPVGMADLVAPEQVKKHYRRAVLAVHPDKAAGQPYEQHAKMIFMELNDAWSEFENQGSRPLF

SEQ ID NO: 45 - protein, 57 aa, Homo Sapien
  ref <308>: DATABASE ACCESSION NUMBER: NCBI/ 2YS8_A
  ref <309>: DATABASE ENTRY DATE: 2020-12-01
  ref <313>: RELEVANT RESIDUES IN SEQ ID NO: (28)..(84)
DSWDMLGVKPGASRDEVNKAYRKLAVLLHPDKCVAPGSEDAFKAVVNARTALLKNIK

SEQ ID NO: 46 - protein, 65 aa, Homo Sapien
  ref <308>: DATABASE ACCESSION NUMBER: NCBI/NP_001035282
  ref <309>: DATABASE ENTRY DATE: 2021-04-19
  ref <313>: RELEVANT RESIDUES IN SEQ ID NO: (51)..(115)
EYYRLLNVEEGCSADEVRESFHKLAKQYHPDSGSNTADSATFIRIEKAYRKVLSHVIEQTNASQS

SEQ ID NO: 47 - protein, 88 aa, Homo Sapien
  ref <308>: DATABASE ACCESSION NUMBER: NCBI/NP_055178
  ref <309>: DATABASE ENTRY DATE: 2021-07-19
  ref <313>: RELEVANT RESIDUES IN SEQ ID NO: (4312)..(4399)
ILKEVTSVVEQAWKLPESERKKIIRRLYLKWHPDKNPENHDIANEVFKHLQNEINRLEKQAFLDQNADRASRRTFSTSASRFQSDKYS

SEQ ID NO: 48 - protein, 66 aa, Homo Sapien
  ref <308>: DATABASE ACCESSION NUMBER: NCBI/NP_115693
  ref <309>: DATABASE ENTRY DATE: 2021-10-03
  ref <313>: RELEVANT RESIDUES IN SEQ ID NO: (49)..(114)
ALYDLLGVPSTATQAQIKAAYYRQCFLYHPDRNSGSAEAAERFTRISQAYVVLGSATLRRKYDRGL

SEQ ID NO: 49 - protein, 6 aa, Artificial
  synthetic tau-binding domain
VQIVYK

SEQ ID NO: 50 - protein, 234 aa, Artificial
  Synthetic tau-binding domain scFv(Tau)
EVQLQQSGAELVQPGASVKLSCTASGFNIKDTSMHWVRQRPEQGLEWIGRIAPANGNTKYDPKFQGKATITTDTSSNTAYLQLSSLTSEDTAVYYCSGSGNYDWGQGTTLTVSGGGGSGGGGSGGGGSDIQMNQSPSSLSASLGDTITISCHASQNINVWLSWYQQKPGNIPKLLIYEASTLYTGVPSRFSGSGSGTGFTLTISSLQPEDIATYYCQQGQSYPWTFGGGTKLEI

SEQ ID NO: 51 - protein, 243 aa, Artificial
  synthetic tau-binding domain scFv(MW7)
QVKLQESGGGLVQPGGSMKLSCAASGFTFSDAWMDWVRQSPEKGLSGVAEIRSKANNHATYYAESVKGRFTISRDDSKSSVYLQMNSLRAEDTGIYYCIYAGFAYWGQGTTVTVSSGGGGSGGGGSGGGGSDIELTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPWTFGGGTKLEI

SEQ ID NO: 52 - protein, 118 aa, Artificial
  synthetic tau-binding domain Happ1
MQSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG

SEQ ID NO: 53 - protein, 6 aa, Artificial
  Synthetic tau-binding domain TBP2
VQIINK

SEQ ID NO: 54 - protein, 13 aa, Artificial
  synthetic tau-binding domain TBP3
YQQYQDATADEQG

SEQ ID NO: 55 - skipped

SEQ ID NO: 56 - protein, 4 aa, Artificial
  Synthetic Linker sequence
GTGS

SEQ ID NO: 57 - protein, 5 aa, Artificial
  Synthetic Linker Sequence
GLESR

SEQ ID NO: 58 - protein, 4 aa, Artificial
  Synthetic Linker Sequence
GGSG

SEQ ID NO: 59 - protein, 4 aa, Artificial
  Synthetic Linker Sequence
GGGS

SEQ ID NO: 60 - protein, 5 aa, Artificial
  Synthetic Linker Sequence
DIAAA

SEQ ID NO: 61 - protein, 9 aa, Artificial
  Synthetic Linker Sequence
DIAAALESR

SEQ ID NO: 62 - protein, 15 aa, Artificial
  Synthetic Linker Sequence
GGGGSGGGGSGGGGS

SEQ ID NO: 63 - protein, 11 aa, Artificial
  Synthetic Linker Sequence
AEAAAKEAAAK

SEQ ID NO: 64 - protein, 15 aa, Artificial
  Synthetic linker sequence
SGGGSGGGGSGGGGS

SEQ ID NO: 65 - protein, 25 aa, Artificial
  Synthetic linker sequence
DIGGGGSGGGGSGGGGSGGGGSAAA

SEQ ID NO: 66 - protein, 232 aa, Artificial
  synthetic epitope to facilitate purification - Human IgG1 Fc domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 8 aa, Artificial
  Synthetic FLAG epitope
DYKDDDDK

SEQ ID NO: 68 - protein, 6 aa, Artificial
  Epitope for purification His6
HHHHHH

SEQ ID NO: 69 - protein, 10 aa, Homo Sapien
  ref <308>: DATABASE ACCESSION NUMBER: NCBI/AAA36340
  ref <309>: DATABASE ENTRY DATE: 1993-04-27
  ref <313>: RELEVANT RESIDUES IN SEQ ID NO: (415)..(424)
EQKLISEEDL

SEQ ID NO: 70 - protein, 9 aa, Human influenza virus
  ref <308>: DATABASE ACCESSION NUMBER: Uniprot/Q03909
  ref <309>: DATABASE ENTRY DATE: 2020-12-02
  ref <313>: RELEVANT RESIDUES IN SEQ ID NO: (119)..(127)
YPYDVPDYA

SEQ ID NO: 71 - protein, 14 aa, Simian virus 5
  ref <301>: AUTHORS: Tomas Hanke, Paul Szawlowski and Richard E. Randall
  ref <302>: TITLE: Construction of solid matrix-antibody-antigen complexes containing simian immunodeficiency virus p27 using tag-specific monoclonal antibody and tag-linked antigen
  ref <303>: JOURNAL: Journal of General Virology
  ref <304>: VOLUME: 73
  ref <306>: PAGES: 653-660
  ref <307>: DATE: 1992
GKPIPNPLLGLDST

SEQ ID NO: 72 - protein, 220 aa, Artificial
  glutathioin S-transferase (GST) tag cloning vector
  Synthetic (1)..(15): Penetrating peptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 73 - protein, 9 aa, human immunodeficiency virus
  ref <308>: DATABASE ACCESSION NUMBER: NCBI/AAF35362
  ref <309>: DATABASE ENTRY DATE: 2013-01-25
  ref <313>: RELEVANT RESIDUES IN SEQ ID NO: (119)..(127)
RKKRRQRRR

SEQ ID NO: 74 - protein, 15 aa, Artificial
  Synthetic cell penetrating peptide
RQIKWFQNRRMKWKK

SEQ ID NO: 75 - protein, 21 aa, Artificial
  Synthetic cell penetrating peptide
KETWWETWWTEWSQPKKKRKV

SEQ ID NO: 76 - protein, 17 aa, Artificial
  Synthetic cell penetrating peptide
CSIPPEVKFNKPFVYLI

SEQ ID NO: 77 - protein, 17 aa, Artificial
  Synthetic signal sequence
MGVKVLFALICIAVAEA

SEQ ID NO: 78 - protein, 19 aa, Artificial
  Synthetic signal sequence
MAPVQLLGLLVLFLPAMRC

SEQ ID NO: 79 - protein, 19 aa, Artificial
  Synthetic signal sequence
MAVLGLLFCLVTFPSCVLS

SEQ ID NO: 80 - protein, 404 aa, Artificial
  V5-Tau(ON4R) tau reporter construct
MGKPIPNPLLGLDSTGTGSEFMAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 81 - protein, 404 aa, Artificial
  V5-Tau(0N4R:P243S) Tau reporter construct
MGKPIPNPLLGLDSTGTGSEFMAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVSGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 82 - protein, 241 aa, Artificial
  Tau3R Tau reporter construct
MIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVD

SEQ ID NO: 83 - protein, 106 aa, Artificial
  JB1-TBP1 fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAVQIVYK

SEQ ID NO: 84 - protein, 147 aa, Artificial
  TBP1-JB1-TBP1 fusion protein construct
MGKPIPNPLLGLDSTGTGSVQIVYKGGGSGGGSGGGSGGGSMGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAVQIVYK

SEQ ID NO: 85 - protein, 128 aa, Artificial
  JB1-2XTBP1 fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAVQIVYKGGGSGGGSGGGSGGGSVQIVYK

SEQ ID NO: 86 - protein, 334 aa, Artificial
  JB1-scFv(Tau) fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAEVQLQQSGAELVQPGASVKLSCTASGFNIKDTSMHWVRQRPEQGLEWIGRIAPANGNTKYDPKFQGKATITTDTSSNTAYLQLSSLTSEDTAVYYCSGSGNYDWGQGTTLTVSGGGGSGGGGSGGGGSDIQMNQSPSSLSASLGDTITISCHASQNINVWLSWYQQKPGNIPKLLIYEASTLYTGVPSRFSGSGSGTGFTLTISSLQPEDIATYYCQQGQSYPWTFGGGTKLEI

SEQ ID NO: 87 - protein, 343 aa, Artificial
  JB1-scFv(MW7 fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAQVKLQESGGGLVQPGGSMKLSCAASGFTFSDAWMDWVRQSPEKGLSGVAEIRSKANNHATYYAESVKGRFTISRDDSKSSVYLQMNSLRAEDTGIYYCIYAGFAYWGQGTTVTVSSGGGGSGGGGSGGGGSDIELTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPWTFGGGTKLEI

SEQ ID NO: 88 - protein, 218 aa, Artificial
  JB1-Happ1 fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAMQSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG

SEQ ID NO: 89 - protein, 106 aa, Artificial
  JB1(P33Q)-TBP fusion protein construct
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAAVQIVYK

SEQ ID NO: 90 - protein, 334 aa, Artificial
  JB1(P33Q)-scFv(Tau) fusion protein construct
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAAEVQLQQSGAELVQPGASVKLSCTASGFNIKDTSMHWVRQRPEQGLEWIGRIAPANGNTKYDPKFQGKATITTDTSSNTAYLQLSSLTSEDTAVYYCSGSGNYDWGQGTTLTVSGGGGSGGGGSGGGGSDIQMNQSPSSLSASLGDTITISCHASQNINVWLSWYQQKPGNIPKLLIYEASTLYTGVPSRFSGSGSGTGFTLTISSLQPEDIATYYCQQGQSYPWTFGGGTKLEI

SEQ ID NO: 91 - protein, 343 aa, Artificial
  JB1(P33Q)-scFv(MW7 fusion protein construct
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAAQVKLQESGGGLVQPGGSMKLSCAASGFTFSDAWMDWVRQSPEKGLSGVAEIRSKANNHATYYAESVKGRFTISRDDSKSSVYLQMNSLRAEDTGIYYCIYAGFAYWGQGTTVTVSSGGGGSGGGGSGGGGSDIELTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPWTFGGGTKLEI

SEQ ID NO: 92 - protein, 218 aa, Artificial
  JB1(P33Q)-Happ1 fusion protein construct
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAAMQSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG

SEQ ID NO: 93 - protein, 91 aa, Artificial
  JB1-QBP1 fusion protein construct
MGKDYYQTLGLARGASDEEIKRAYRRQALRYHPDKNKEPGAEEKFKEIAEAYDVLSDPRKREIFDRYGEEGLKGSGGGGSSNWKWWPGIFD

SEQ ID NO: 94 - protein, 91 aa, Artificial
  JB1(P33Q)-QBP1 fusion protein construct
MGKDYYQTLGLARGASDEEIKRAYRRQALRYHQDKNKEPGAEEKFKEIAEAYDVLSDPRKREIFDRYGEEGLKGSGGGGSSNWKWWPGIFD

SEQ ID NO: 95 - protein, 106 aa, Artificial
  JB1-TBP2 fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAVQIINK

SEQ ID NO: 96 - protein, 113 aa, Artificial
  JB1-TBP3 fusion protein construct
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAYQQYQDATADEQG

SEQ ID NO: 97 - protein, 86 aa, Artificial
  JB1-TBP1 (no linker) fusion protein construct
MGKDYYQTLGLARGASDEEIKRAYRRQALRYHPDKNKEPGAEEKFKEIAEAYDVLSDPRKREIFDRYGEEGLKGSGGGGSVQIVYK

SEQ ID NO: 98 - protein, 86 aa, Artificial
  JB1-TBP2 (no linker) fusion protein construct
MGKDYYQTLGLARGASDEEIKRAYRRQALRYHPDKNKEPGAEEKFKEIAEAYDVLSDPRKREIFDRYGEEGLKGSGGGGSVQIINK

SEQ ID NO: 99 - protein, 93 aa, Artificial
  JB1-TBP3 (no linker) fusion protein construct
MGKDYYQTLGLARGASDEEIKRAYRRQALRYHPDKNKEPGAEEKFKEIAEAYDVLSDPRKREIFDRYGEEGLKGSGGGGSYQQYQDATADEQG

SEQ ID NO: 100 - protein, 314 aa, Artificial
  JB1-scFv(Tau) (no linker) fusion protein construct
MGKDYYQTLGLARGASDEEIKRAYRRQALRYHPDKNKEPGAEEKFKEIAEAYDVLSDPRKREIFDRYGEEGLKGSGGGGSEVQLQQSGAELVQPGASVKLSCTASGFNIKDTSMHWVRQRPEQGLEWIGRIAPANGNTKYDPKFQGKATITTDTSSNTAYLQLSSLTSEDTAVYYCSGSGNYDWGQGTTLTVSGGGGSGGGGSGGGGSDIQMNQSPSSLSASLGDTITISCHASQNINVWLSWYQQKPGNIPKLLIYEASTLYTGVPSRFSGSGSGTGFTLTISSLQPEDIATYYCQQGQSYPWTFGGGTKLEI

SEQ ID NO: 101 - protein, 198 aa, Artificial
  JB1-Happ1 (no linker) fusion protein construct
MGKDYYQTLGLARGASDEEIKRAYRRQALRYHPDKNKEPGAEEKFKEIAEAYDVLSDPRKREIFDRYGEEGLKGSGGGGSMQSVLTQPPSASGTPGQRVTISCSGSSSNIGSNYVYWYQQLPGTAPKLLIYRNNQRPSGVPDRFSGSKSGTSASLAISGLRPEDEADYYCAAWDDSLCVALVFGGGTNGGGGVDGTAG